(19) United States
(12) Patent Application Publication
(10) Pub. No.: US 2024/0306985 A1
(43) Pub. Date: Sep. 19, 2024

(54) MODULAR DISEASE MANAGEMENT DEVICE AND AUTOMATED NEEDLE AND CANNULA INSERTION DEVICE

(71) Applicant: Willow Laboratories, Inc., Irvine, CA (US)

(72) Inventors: Hung The Vo, Fountain Valley, CA (US); Sai Kong Frank Lee, Irvine, CA (US); Tuan Minh Tran, Garden Grove, CA (US); Peixuan Zhang, Ontario, CA (US); Pouya Jalalmanesh, San Pedro, CA (US); Amir K. Dabiri-Asgari, Mission Viejo, CA (US)

(21) Appl. No.: 18/605,630

(22) Filed: Mar. 14, 2024

Related U.S. Application Data
(60) Provisional application No. 63/490,601, filed on Mar. 16, 2023.

(51) Int. Cl. A61B 5/00

(52) U.S. Cl. CPC A61B 5/4839

(57) ABSTRACT

A device may include a base configured to at least partially couple to skin of a patient. A device may include at least one module configured to removably couple to the base, the at least one module can include a first module. The first module can include a medication bladder, a medication pump configured to cause medication to flow from the medication bladder through a cannula configured to be inserted into a tissue site of the patient; and a cannula insertion device configured to insert the cannula into the tissue site of the patient by actuation of a spring released by a triggering component.

### FIELD OF THE DISCLOSURE

The general field of this disclosure is glucose sensing and disease management systems.

### BACKGROUND

Diabetes is a chronic disease that impacts many individuals, both adults and children. The management of diabetes may include the measurement of glucose within the interstitial space including blood and/or interstitial fluid of a patient and administration of insulin to the patient. A closed loop insulin administration system includes both a sensor to take glucose measurements from the interstitial space including blood and/or interstitial fluid of the patient and an insulin administration device which administers insulin to the patient based on the glucose measurements. Closed loop insulin administration systems allow individuals impacted by diabetes to go about daily life with much less worry about their insulin or glucose levels which can vastly improve a diabetic's quality of life.

### SUMMARY OF THE INVENTION

Various embodiments of systems, methods and devices within the scope of the appended claims each have several aspects, no single one of which is solely responsible for the desirable attributes described herein. Without limiting the scope of the appended claims, some prominent features are described herein.

In some embodiments, the systems, methods and devices described herein can include a modular disease management system. The modular disease management system can include a base configured to at least partially couple to skin of a patient. The modular disease management system can include at least one module configured to removably couple to the base. The at least one module can include a first module. The first module can include a medication bladder, a medication pump configured to cause medication to flow from the medication bladder through a cannula configured to be inserted into a tissue site of the patient, and a cannula insertion device configured to insert the cannula into the tissue site of the patient by actuation of a spring released by a triggering component.

In some embodiments, the systems, methods and devices described herein can include a disease management system. The disease management system can include a medication bladder. The disease management system can include a medication pump configured to cause medication to flow from the medication bladder through a cannula configured to be inserted into a tissue site of a patient. The disease management system can include a cannula insertion device configured to insert the cannula into the tissue site of the patient. The cannula insertion device can include a needle, at least one spring, and a triggering component. The triggering component can include a trigger pin configured to release a first spring when actuated by a trigger arm, and a nitinol wire configured to contract in response to an electrical signal, causing the trigger arm to actuate the trigger pin. In some embodiments, the first spring, when released, is configured to insert the cannula into the tissue site of the patient. The disease management system can include a controller including one or more processors electrically coupled to the cannula insertion device and configured to transmit the electrical signal to the nitinol wire.

In some embodiments, the systems, methods and devices described herein can include an automatic insertion device. The automatic insertion device can include a needle configured to insert a device at a tissue site of a patient when actuated, one or more springs configured to actuate the needle when released, and a triggering component configured to release the one or more springs. The triggering component can include a trigger pin configured to release at least a first spring of the one or more springs when actuated by a trigger arm, and a nitinol wire configured to contract in response to an electrical signal, causing the trigger arm to actuate the trigger pin. In some embodiments, upon the nitinol wire a contracting, the triggering component releases at least the first spring, the first spring actuates the needle, and the device is inserted at the tissue site of the patient.

In some embodiments, the systems, methods and devices described herein can include a method for automatically inserting a device into a patient. The method can include inserting a cannula into a needle configured to insert the cannula when the needle is inserted at a tissue site of a patient. The method can include activating, using an electrical signal, a triggering component configured to release one or more springs, at least a first spring of the one or more springs configured to insert the needle into the tissue site. The triggering component can include a trigger pin configured to release at least the first spring when actuated by a trigger arm, and a nitinol wire configured to contract in response to the electrical signal. The method can include causing the trigger arm to actuate the trigger pin; and administering, using a medication pump coupled to the cannula, a medication to the patient via the cannula.

In some embodiments, the systems, methods and devices described herein can include a modular disease management system, including: a base configured to receive a plurality of modules, the base including: an electrical connector; an adhesive layer; and at least one physical interface, wherein each physical interface is configured to couple the plurality of modules to a surface of the base and to provide an electrical signal to the plurality of modules via the electrical connector; wherein, the plurality of modules include: a controller module; a pump module, wherein the pump module includes a pump and a pouch assembly containing a substance, wherein the pouch assembly is configured to connect with the pump; at least one sensor module; and at least one medication module; and wherein, the controller module is configured to activate the pump such that the pump draws the substance from the pouch assembly into the medication module.

In some embodiments, the base, the at least one sensor module, the at least on medication module, and/or the pump module are disposable. In some embodiments, the at least one sensor module includes a continuous glucose monitor injector and the at least one medication module includes an insulin injector. In some embodiments, the continuous glucose monitor injector is configured to send a glucose reading to the controller module. In some embodiments, the continuous glucose monitor injector and the insulin injector are housed in a single automatic insertion module. In some embodiments, the controller module includes a battery and a control unit. In some embodiments, the battery is configured to allow for volume expansion. In some embodiments, the controller module includes at least one cutout for a high profile component attached to the control unit. In some embodiments, the controller module is configured to communicate with a user terminal. In some embodiments, the electrical connector is a flexible cable. In some embodiments, the flexible cable is 0.31 millimeters thick. In some embodiments, the controller module includes a compartment seal configured to interface with the physical interface to create an electrical contact with the electrical connector and prevent water ingress into the controller module.

In some embodiments, the systems, methods and devices described herein can include an automatic insertion device for inserting a cannula into a patient, including: a housing; a needle associated with a cannula positioned in the housing; a launch spring holder configured to house a launch spring, the launch spring configured to push the needle and cannula forward; a first trigger release arm configured to apply an opposing force on the launch spring holder such that the launch spring is in a hold position; and a second trigger release arm coupled to the first trigger release arm, the second trigger release arm further coupled to an actuator configured to contract in response to an electrical signal such that the first trigger release arm and the second trigger release arm withdraw when the actuator contracts; and wherein, upon the withdrawal of the first trigger release arm and the second trigger release, the opposing force on the launch spring holder is disengaged such that the launch spring pushes the needle and cannula forward to allow for the needle and the cannula to insert into a patient.

In some embodiments, the actuator is a nitonol wire. In some embodiments, the device further includes at least one guiding rail located at a top of the housing. In some embodiments, the first trigger release arm includes a rectangular carve out configured to allow the second trigger release arm to connect to the first trigger release arm. In some embodiments, the second trigger release arm includes an end that is circular in shape, such that the second trigger release arm is held under tension by the rectangular carve out prior to the actuator contracting and the second trigger release is able to freely rotate with the rectangular carve out during the actuator contracting. In some embodiments, the second trigger release arm is rectangular in shape. In some embodiments, the needle is U-shaped such that the cannula is located inside the U-shape of the needle. In some embodiments, the automatic insertion device for inserting a cannula into a patient may further include a retract spring holder configured to house a retract spring in a hold position; and a release switch configured to release the retract spring holder; wherein, upon the insertion of the needle and cannula into the patient, the release switch releases the retract spring holder such that the retract spring removes the needle from the patient while the cannula remains inserted into the patient.

In some embodiments, the systems, methods and devices described herein can include an automatic insertion device for inserting an analyte sensor into a patient, including: a housing; a needle associated with an analyte sensor positioned in the housing; a launch spring holder configured to house a launch spring, the launch spring configured to push the needle and the analyte sensor forward; a first trigger release arm configured to apply an opposing force on the launch spring holder such that the launch spring is in a hold position; and a second trigger release arm coupled to the first trigger release arm, the second trigger release arm further coupled to an actuator configured to contract in response to an electrical signal such that the first trigger release arm and the second trigger release arm withdraw when the actuator contracts; and wherein, upon the withdrawal of the first trigger release arm and the second trigger release, the opposing force on the launch spring holder is disengaged such that the launch spring pushes the needle and cannula forward to allow for the needle and the cannula to insert into a patient.

In some embodiments, the systems, methods and devices described herein can include a method for automatically inserting a cannula into a patient, the method including: preloading a launch spring configured to push a needle and a cannula forward along a fixed axis, wherein the launch spring is housed in a launch spring holder and wherein a first trigger release arm is configured to apply an opposing force on the launch spring holder such that the launch spring is in a hold position, wherein a second trigger release arm is coupled to the first trigger release arm, and the second trigger release arm is further coupled to an actuator configured to contract in response to an electrical signal such that the first trigger release arm and the second trigger release arm withdraw when the actuator contracts, and wherein, upon the withdrawal of the first trigger release arm and the second trigger release, the opposing force on the launch spring holder is disengaged such that the launch spring pushes the needle and cannula forward to allow for the needle and the cannula to insert into the patient; and causing the actuator to retract.

In some embodiments, the method for automatically inserting a cannula into a patient can further include preloading a retract spring configured to push the needle backward along the fixed axis, wherein the retract spring is housed in a retract spring holder and wherein a retract release trigger is configured to apply an opposing force on the retract spring holder such that the retract spring is in a hold position; wherein, upon the insertion of the needle and cannula into the patient, the retract release trigger releases the retract spring holder such that the retract spring removes the needle from the patient while the cannula remains inserted into the patient.

In some embodiments, the systems, methods and devices described herein can include a method for automatically inserting an analyte sensor into a patient, the method including: preloading a launch spring configured to push a needle and an analyte sensor forward along a fixed axis, wherein the launch spring is housed in a launch spring holder and wherein a first trigger release arm is configured to apply an opposing force on the launch spring holder such that the launch spring is in a hold position, wherein a second trigger release arm is coupled to the first trigger release arm, and the second trigger release arm is further coupled to an actuator configured to contract in response to an electrical signal such that the first trigger release arm and the second trigger release arm withdraw when the actuator contracts, and wherein, upon the withdrawal of the first trigger release arm and the second trigger release, the opposing force on the launch spring holder is disengaged such that the launch spring pushes the needle and the analyte sensor forward to allow for the needle and the analyte sensor to insert into the patient; and causing the actuator to retract.

In some embodiments, the method for automatically inserting an analyte sensor into a patient can further include preloading a retract spring configured to push the needle backward along the fixed axis, wherein the retract spring is housed in a retract spring holder and wherein a retract release trigger is configured to apply an opposing force on the retract spring holder such that the retract spring is in a hold position; wherein, upon the insertion of the needle and cannula into the patient, the retract release trigger releases the retract spring holder such that the retract spring removes the needle from the patient while the analyte sensor remains inserted into the patient.

In some embodiments, the systems, methods and devices described herein can include an automatic insertion device, including: a housing; a needle associated with a cannula located within the housing; at least three torsion springs configured to connect to an actuator located within the housing; at least three spring holders configured to preload each torsion spring; and the actuator configured to release the at least three torsion springs upon a trigger.

In some embodiments, the at least three torsion springs includes: a first spring; a second spring; and a third spring, wherein the actuator is configured to release the first spring which triggers the release of the second spring and then releases the needle and cannula forward to insert into a patient, wherein the third spring is configured to retrieve the needle from insertion, and wherein the third spring is further configured to hold the needle at a high position. In some embodiments, a guide rail holds the needle and the cannula at an angle. In some embodiments, the actuator is a nitinol wire. In some embodiments, the actuator is an electrical actuator. In some embodiments, the at least torsion springs remain in a locked position until the trigger.

In some embodiments, the systems, methods and devices described herein can include a method for automatically inserting a cannula into a patient, the method including: preloading a first torsion spring, wherein the first torsion spring is held at tension by a first spring holder and a trigger release holder; preloading a second torsion spring, wherein the second torsion spring is held at tension by a second spring holder and a needle holder, wherein the needle holder is held in place by a trigger; preloading a third torsion spring, wherein the third torsion spring is held at tension by a third spring holder and a guiderail; and causing the first torsion spring to disengage from the trigger release holder, wherein, upon the first torsion spring disengaging from the trigger release holder, the first torsion spring causes the trigger to rotate and disengage from the second torsion spring, wherein, upon the trigger disengaging from the second torsion spring, the second torsion spring drives the needle holder forward along the guiderail and a needle into the patient, wherein, upon the needle being inserted into the patient, the third torsion spring is disengaged from the guiderail, wherein, upon the third torsion spring disengaging from the guiderail, the third torsion spring drives the needle holder backward along the guiderail and the needle out of the patient.

In some embodiments, the systems, methods and devices described herein can include method for automatically inserting a cannula into a patient, the method including: preloading a launch spring configured to push needle carrier, coupled to a needle, and a cannula carrier, coupled to a cannula, forward along at least one guiderail, wherein the launch spring is coupled to a launch spring holder configured to rotate in response to a downward force and wherein a trigger release is configured to apply an opposing force on the launch spring holder such that the launch spring is in a hold position and the launch spring holder is in a hold position, wherein the trigger release is coupled to an actuator configured to contract in response to an electrical signal such that the trigger release withdraws when the actuator contracts, wherein, upon the withdrawal of the trigger release, the opposing force on the launch spring holder is disengaged such that the launch spring pushes the needle carrier and cannula carrier forward along the at least one guiderail to allow for the needle and the cannula to insert into the patient; and causing the actuator to retract.

In some embodiments, the method for automatically inserting a cannula into a patient can further include preloading a retract spring configured to push the needle carrier backward along the at least one guiderail, wherein the retract spring is housed in a retract spring holder and wherein a retract release trigger is configured to apply an opposing force on the retract spring holder such that the retract spring is in a hold position; wherein, upon the insertion of the needle and cannula into the patient, a rotator spring applies a force on the launch spring holder such that the launch spring holder rotates, wherein, upon the launch spring holder rotating, the launch spring pushes the needle carrier such that the needle carrier disengages with the at least one guiderail, and wherein, upon the needle carrier disengaging from the at least one guiderail, the retract release trigger releases the retract spring holder such that the retract spring pushes the needle carrier backward and the needle out of the patient.

In some embodiments, the systems, methods and devices described herein can include an automatic insertion device for inserting a cannula into a patient, including: a housing; a needle associated with a cannula positioned in the housing; at least one guide rail; a launch spring holder configured to house a launch spring, the launch spring configured to push the needle and cannula forward along the guide rail; a trigger release configured to hold the launch spring in a tension state; an actuator coupled to the trigger release, the actuator configured to retract the trigger release; and where, upon the actuator retracting the trigger release, the trigger release disengages from the launch spring such that the launch spring pushes the needle and cannula forward to allow for the needle and the cannula to insert into a patient.

In some embodiments, the launch spring holder is further configured to rotate within the housing. In some embodiments, the automatic insertion device can further include a stamp sheet metal spring configured to cause the launch spring holder to rotate into alignment with the guide rail. In some embodiments, the automatic insertion device can further include a retraction spring configured to engage with the needle; wherein, upon the insertions of the needle and the cannula into a patient, the retraction spring is released such that the needle is pushed backward along the guide rail and retracted from the patient. In some embodiments, the at least one guide rail includes a guide wire. In some embodiments, the retraction spring is an elastic band. Systems and methods disclosed herein relate to a modular disease management system that may include a sensor module, a pump module, a medication module, and a controller module. Each module may be configured to allow some modules to be removed and disposed of and other modules reused. The sensor module and the medication module may include an automatic insertion device configured to automatically insert a cannula or analyte sensor into a patient.

### BRIEF DESCRIPTION OF THE DRAWINGS

These and other features, aspects, and advantages of the present application are described with reference to drawings of certain embodiments, which are intended to illustrate, but not limit, the present disclosure. It is to be understood that the attached drawings are for the purpose of illustrating concepts disclosed in the present application and may not be to scale:

FIG. 1 illustrates an example disease management system that may be part of a disease management environment or used as an interleaved device.

FIG. 2A illustrates a perspective view of an example modular disease management device.

FIG. 2B is a top view of an example modular disease management device.

FIG. 2C is a side view of an example modular disease management device.

FIG. 2D is a bottom view of an example modular disease management device.

FIG. 2E is a perspective cross section view of an example modular disease management device.

FIG. 2F illustrates an exploded view of the components of an example controller module.

FIG. 2G illustrates an example process of medication administration by a modular disease management device.

FIG. 3 illustrates a perspective view of an alternate example modular disease management device.

FIG. 4A illustrates a perspective view a second alternate example modular disease management device.

FIG. 4B illustrates a side view of the second alternate example modular disease management device.

FIG. 4C illustrates a bottom view of the second alternate example modular disease management device.

FIG. 4D illustrates an exploded view of the second alternate example modular disease management device.

FIG. 4E illustrates a top view of an example base for a modular disease management device.

FIG. 4F illustrates a bottom view of an example base for a modular disease management device.

FIG. 4G illustrates a side view of an example base for a modular disease management device.

FIGS. 4H and 4I illustrate side views of an example disposable module for a modular disease management device.

FIG. 4J illustrates a bottom view of an example disposable module for a modular disease management device.

FIG. 4K illustrates a top view of an example disposable module for a modular disease management device.

FIG. 4L illustrates a bottom view of an example disposable module for a modular disease management device.

FIG. 4M illustrates a side view of an example reusable module for a modular disease management device.

FIG. 4N illustrates an exploded view of an example reusable module for a modular disease management device.

FIG. 4O illustrates a top view of an example controller module for a modular disease management device.

FIG. 4P illustrates bottom view of an example reusable module for a modular disease management device.

FIG. 5 illustrates an example process for a user utilizing a modular disease management system.

FIG. 6A illustrates a perspective view of an example automated needle and cannula insertion and needle removal device.

FIG. 6B is a cross section view for an example automated needle and cannula insertion and needle removal device.

FIG. 6C illustrates an example process of a controller using an automated needle and cannula insertion and needle removal device.

FIG. 6D illustrates an example operation process for an automated needle and cannula insertion and needle removal device.

FIG. 7A illustrates a perspective view of an alternate example automated needle and cannula insertion and needle removal device prior to launch sequence.

FIG. 7B is a perspective view of an alternate example automated needle and cannula insertion and needle removal device during launch sequence.

FIG. 7C is a cross section view of an alternate example automated needle and cannula insertion and needle removal device during launch sequence.

FIG. 7D is a perspective view of an alternate example automated needle and cannula insertion and needle removal device during retract sequence.

FIG. 7E is a cross section view of an alternate example automated needle and cannula insertion and needle removal device during retract sequence.

FIG. 7F illustrates an alternate example operation process for an automated needle and cannula insertion and needle removal device.

FIG. 8A illustrates a perspective view of a second alternate example automated needle and cannula insertion and needle removal device.

FIG. 8B is a side view of a second alternate example automated needle and cannula insertion and needle removal device.

FIG. 8C is a cross section view of a second alternate example automated needle and cannula insertion and needle removal device prior to launch sequence.

FIG. 8D is a cross section view of a second alternate example automated needle and cannula insertion and needle removal device during launch sequence.

FIG. 8E illustrates a second alternate example operation process for an automated needle and cannula insertion and needle removal device.

FIG. 9A illustrates a side view of a third alternate example automated needle and cannula insertion and needle removal device.

FIG. 9B illustrates a perspective view of a third alternate example automated needle and cannula insertion and needle removal device.

FIG. 9C illustrates a cross section view of a third alternate example automated needle and cannula insertion and needle removal device.

FIG. 10A illustrates a side view of an example automated needle and sensor insertion and needle removal device.

FIG. 10B illustrates a cross section side view of an example automated needle and sensor insertion and needle removal device.

FIG. 10C illustrates a cross section front view of an example automated needle and sensor insertion and needle removal device.

FIG. 10D illustrates a cross section top view of an example automated needle and sensor insertion and needle removal device.

FIG. 11A illustrates a side view of a fourth alternate example automated needle and cannula insertion and needle removal device.

FIG. 11B illustrates a cross section side view of a fourth alternate example automated needle and cannula insertion and needle removal device.

FIG. 11C illustrates a cross section top view of a fourth alternate example automated needle and cannula insertion and needle removal device.

FIG. 12A illustrates a side and back view of an example retracting wire configuration for an automated needle insertion and needle removal device.

FIG. 12B illustrates a side and bottom view of an alternative example retracting wire configuration for an automated needle insertion and needle removal device.

FIG. 12C illustrates an example process for an automated needle insertion and needle removal device.

FIG. 13A illustrates a cross section side view of an automated needle insertion and needle removal device prior to launch.

FIG. 13B illustrates a cross section side view of an automated needle insertion and needle removal device with a trigger arm rotated.

FIG. 13C illustrates a cross section side view of an automated needle insertion and needle removal device with a needle in an inserted position.

FIG. 13D illustrates a cross section side view of an automated needle insertion and needle removal device with a needle in a retracted position.

FIG. 13E illustrates an example process for an automated needle insertion and needle removal device.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims that may arise here from is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Example Disease Management System

As described above, closed loop medication administration systems, such as closed loop insulin administration systems, can improve the quality of life of a patient who requires regular administration of medication and monitoring of various physiological and/or other parameters. The patient's quality of life can be further improved as more components of and/or supporting a closed loop medication administration system are incorporated into a disease management system.

In various implementations, a disease management system can include some or all components of a closed loop medication administration system in a self-contained unit. The disease management system may be applied on a patient allowing for case of installation and removal of the disease management system. However, other applications may also be possible.

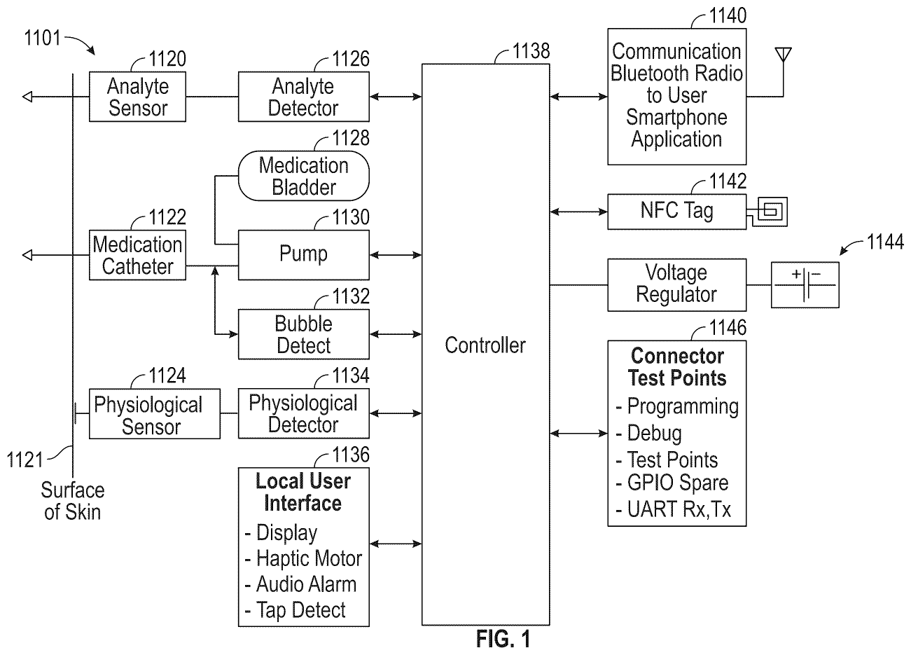

FIG. 1 shows a block diagram of an example disease management system 1101. In some examples, the disease management system 1101 may be part of a disease management environment, such as the closed-loop diabetes management environment described above. A disease management system 1101 may be configured to measure one or more physiological parameters of a patient (such as pulse, skin temperature, or other values), measure one or more analytes present in the blood of a patient (such as glucose, lipids, or other analytes) and administer medication (such as insulin, glucagon, or other medication). In some examples, a disease management system 1101 may be configured to communicate with one or more hardware processors that may be external to the disease management system 1101, such as a cloud-based processor or user device. A disease management system 1101 may include near-field communication (NFC) tag to support authentication and pairing with a user device (for example, smart phone or smart watch), Bluetooth communication with additional disease management systems or devices, and Bluetooth communication with a paired user device running an associated control application. To support ease of use and safe interaction with the patient, the system may incorporate user input through a tap-detecting accelerometer (also referred to herein as a “tap detector”) and provide feedback via an audio speaker, haptic vibration, and/or optical indicators. The system may operate on battery power and support both shelf-life and reliable operation once applied to the patient. Battery life may be managed through control of several planned levels of sleep and power consumption. To support this reliability, a controller can monitor several system-health parameters, and monitor temperatures of the included medication, and ambient temperature for the life of the device.

As illustrated in FIG. 1, a controller 1138 of the disease management system 1101 may be configured to communicate and control one or more components of the disease management system 1101. The controller 1138 may include one or more hardware processors, printed circuit boards (PCBs), integrated circuits (ICs), application specific integrated circuits (ASICs), and/or the like. The controller 1138 may be configured to communicate with peripheral devices or components to support the accurate measurement of physiological parameters and blood analytes, such as patient pulse, temperature, and blood glucose, using detector electronics. The controller 1138 may subsequently calculate dose or receive a calculated dose value and administer medication. For example, the controller 1138 may calculate or receive a dose value of insulin and administer the dose of insulin via an actuated pump. The controller 1138 may record device activity and transfer the recorded data to non-volatile secure memory space. At the end of the life of a device or system, the controller can be configured to lock operation, and create a data recovery module to permit authenticated access to the recorded data if needed.

A disease management system 1101 may include an analyte sensor 1120. The analyte sensor 1120 may be configured to detect analytes in the patient's blood. For example, an analyte sensor 1120 can include a glucose sensing probe configured to pierce the surface of the skin 1121. In some implementations, the analyte sensor 1120 may be an electrochemical analyte sensor, an optical sensor, or another sensor used to measure an analyte. In some examples, a disease management system 1101 may include a plurality of analyte sensors 1120 to detect one or more analytes. In some examples, an analyte sensor 1120 may be configured to detect a plurality of analytes. Sensed analytes may include, but are not limited to, glucose, insulin, and other analytes. An analyte sensor 1120 may be configured to communicate with an analyte detector 1126. The analyte detector 1126 may be configured to receive a signal of one or more analyte sensors 1120 in order to measure one or more analytes in the blood of the patient. The analyte detector 1126 may be configured to communicate with the controller 1138. For example, the analyte detector 1126 may be configured to, for example, send analyte values to the controller 1138 and receive control signals from the controller. In some examples, the analyze sensor 1120, analyte detector 1126, and/or controller 1138 may comprise a continuous glucose monitor (CGM) system.

A disease management system 1101 may include a medication catheter 1122. The medication catheter 1122 may be configured to administer medication or other fluid, including, but not limited to insulin, glucagon, or other medication, to the patient. The medication catheter 1122 may receive medication from a medication bladder 1128 configured to contain medication to be administered. The medication bladder 1128 may be configured to contain medication for a prolonged period, such as one or more days. For example, 1, 3, 6, or more days. The medication bladder 1128 may be configured to contain certain medication types, such as insulin, glucagon, or other medication. In some examples, a disease management system 1101 may include a plurality of medication bladders 1128 for one or more reservoirs of the same or different medications. In some examples, a disease management system 1101 may be configured to mix medications from medication bladders 1128 prior to administration to the patient. A pump 1130 may be configured to cause medication to be administered from the bladder 1128 to the patient through the insulin catheter 1122. A pump 1130 may include, but is not limited to, a pump such as described herein.

A disease management system 1101 may optionally include a physiological sensor 1124. The physiological sensor 1124 may include a pulse rate sensor, temperature sensor, pulse oximeter, the like or a combination thereof. In some examples, a disease management system 1101 may be configured to include a plurality of physiological sensors. The physiological sensor 1124 may be configured to communicate with a physiological detector 1134. The physiological detector 1134 may be configured to receive a signals of the physiological sensor 1124. The physiological detector 1134 may be configured to measure or determine and communicate a physiological value from the received signals. The physiological detector 1134 may be configured to communicate with the controller 1138. For example, the physiological detector 1134 may be configured to send measured physiological values to the controller 1138 and receive control signals from the controller.

A disease management system 1101 may include one or more local user interfacing components 1136. For example, the local user interfacing components 1136 may include, but are not limited to one or more optical displays, haptic motors, audio speakers, and user input detectors. In some examples, an optical display may include an LED light configured to display a plurality of colors. In some examples, an optical display may include a digital display of information associated with the disease management system 1101, including, but not limited to, device status, medication status, patient status, measured analyte or physiological values, the like or a combination thereof. In some examples, a user input detector may include an inertial measurement unit, tap detector, touch display, or other component configured to accept and receive user input. In some examples, audio speakers may be configured to communicate audible alarms related to device status, medication status user status, the like or a combination thereof. A controller 1138 may be configured to communicate with the one or more local interfacing components 1136 by, for example, receiving user input from the one or more user input components or sending control signals to, for example, activate a haptic motor, generate an output to the optical display, generate an audible output, or otherwise control one or more of the local user interfacing components 1136.

A disease management system 1101 may include one or more communication components 1140. The communication components 1140 can include, but are not limited to, one or more radios configured to emit Bluetooth, cellular, Wi-Fi, or other wireless signals. In some examples, the communication components 1140 can include a port for a wired connection. Additionally, a disease management system 1101 may include an NFC tag 1142 to facilitate in communicating with one or more hardware processors. The communication components 1140 and NFC tag 1142 may be configured to communicate with the controller 1138 in order to send and/or receive information associated with the disease management system 1101. For example, a controller 1138 may communicate medication information and measured values through the communication components 1140 to an external device. Additionally, the controller 1138 may receive instructions associated with measurement sampling rates, medication delivery, or other information associated with operation of the disease management system 1101 through the communication components 1140 from one or more external devices.

A disease management system 1101 may include one or more power components 1144. The power components may include, but are not limited to, one or more batteries and/or other power sources and power management components, such as voltage regulators, power conversion circuitry and the like. Power from the power components 1144 may be accessed by the controller and/or other components of the disease management system 1101 to operate the disease management system 1101.

A disease management system 1101 may have one or more power and sleep modes to help regulate power usage. For example, a disease management system 1101 may have a sleep mode. The sleep mode may be a very low power mode with minimal functions, such as the real time clock (RTC) and alarms that can wake the system and perform tasks, such as taking a temperature measurement of the system, or the like. In another example, a disease management system 1101 may include a measure temperature mode which may correspond to a low power mode with reduced functions. The measure temperature mode may be triggered by the RTC where the system is configured to take a temperature measurement, save the value, and return the system to a sleep mode. In another example, a disease management system 1101 may include a wake up mode. The wake up mode may be triggered by an NFC device and allow the system to pair with an external device with, for example, Bluetooth. If a pairing event does not occur, the system may return to sleep mode. In another example, a disease management system 1101 may include a pairing mode. The pairing mode may be triggered by an NFC device. When a controlling application is recognized, the system may proceed to pair with the application and set the system to an on condition and communicate to the cloud or other external device to establish initial data movement. In another example, a disease management system 1101 may include a rest mode where the system is configured to enter a lower power mode between measurements. In another example, a disease management system 1101 may include a data acquisition mode where the system is configured to enter a medium power mode where data acquisition takes place. In another example, a disease management system 1101 may include a parameter calculation mode where the system is configured to enter a medium power mode where parameter calculations, such as a blood glucose calculations, are performed and data is communicated to an external device and/or the cloud. In another example, a disease management system 1101 may include a pump mode where the system is configured to enter a higher power mode where the pump draws power to deliver medication to the patient.

A disease management system 1101 may include one or more connector test points 1146. The connecter test points may be configured to aid in programming, debugging, testing or other accessing of the disease management system 1101. In some examples, connector test points 1146 may include, for example, general purpose input-output (GPIO) connections, universal asynchronous receiver/transmitter (UART) connections, the like or a combination thereof.

### Example Modular Disease Management System

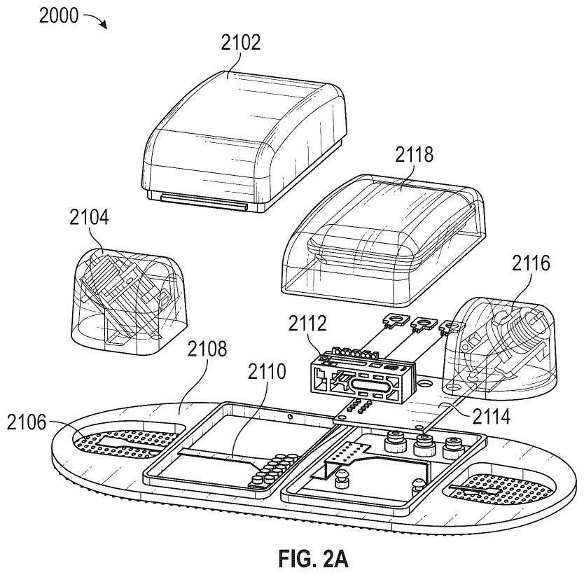

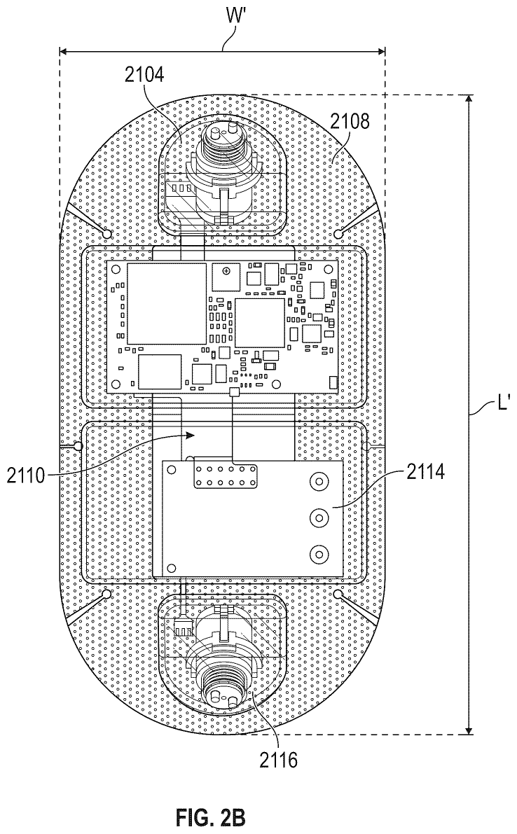

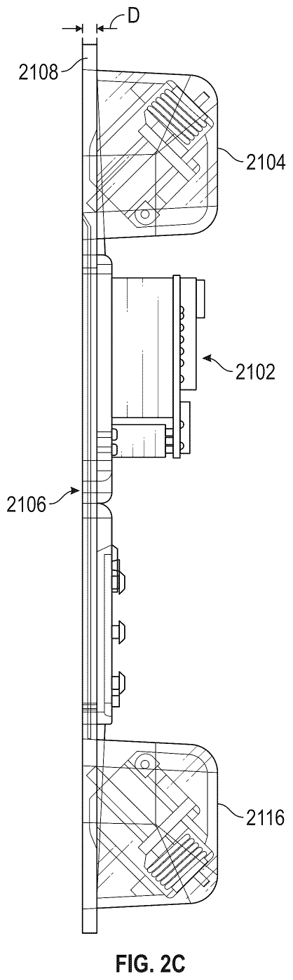

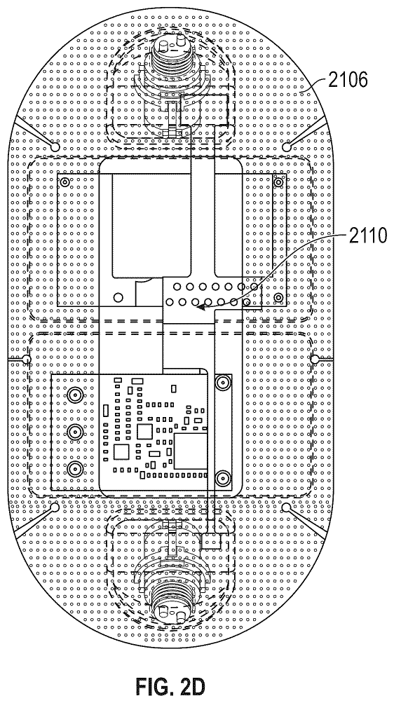

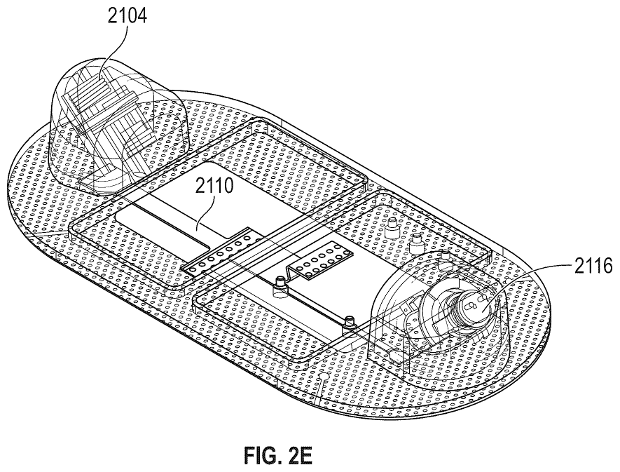

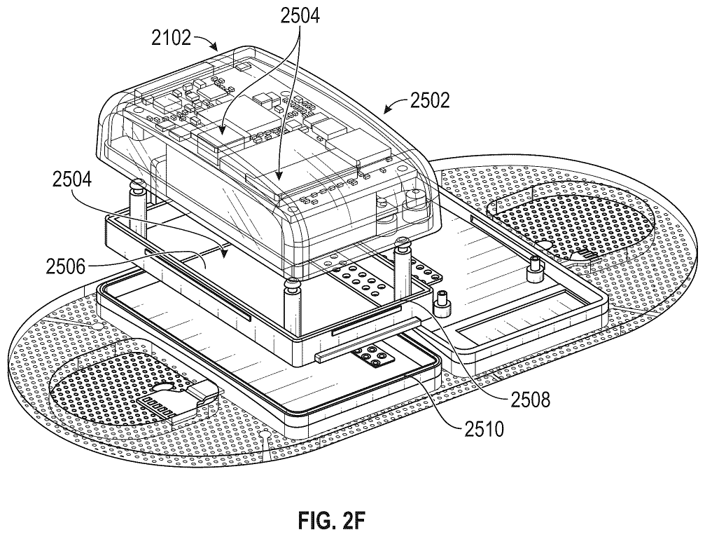

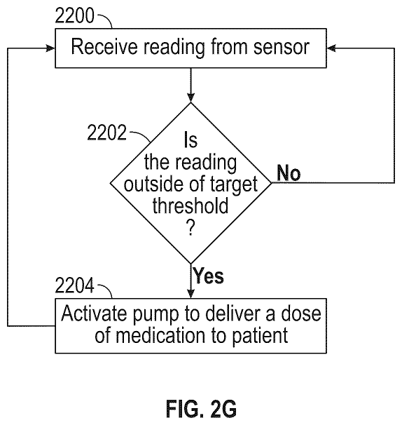

FIGS. 2A-2F illustrate an example of a modular disease management system 2000. FIGS. 2A-2F depict different scenarios that all share the same features and thus identically labeled elements share the identical description as the description provided in connection with FIG. 2A.

Disease management system 2000 can include any one or more of the features discussed above with respect to the disease management system 1101 in addition or in the alternative to the features described below. In some examples, the disease management system 2000 may have alternative or different features than described above with respect to the disease management system 1101. Disease management system 2000 may be used by a patient to monitor and/or manage one or more diseases. For example, disease management system 2000 may be configured to measure one or more physiological parameters of a patient (such as pulse, skin temperature, or other values), measure one or more analytes present in the blood of a patient (such as glucose, lipids, or other analytes) and administer medication (such as insulin, glucagon, or other medication) to a patient.

As described above, incorporating more components used for and/or to support a closed loop medication administration system into a self-contained unit, such as a disease management system 2000 or a disease management system 1101 can be beneficial. However, in some implementations, different components of a disease management system can have different lifespans. For example, the medication store, such as the medication bladder 1128, may run out before the disease management system requires replacement. In some implementations, some components may not be easily disposable and/or may be expensive to replace. For example, a controller component, such as controller 1138, may be expensive to replace. However, the disease management system may require periodic replacement (e.g., periodically replacing the disease management system may help prevent infection or other conditions). As such, it may be advantageous to remove and/or disconnect the controller component from one disease management system that was removed from a patient and use the controller component in another disease management system is installed on the patient as a replacement. Advantageously, the present disease management system 2000 can contain modules that can be independently removed and/or replaced to extend the overall life of the disease management system 2000 and/or be removed and/or inserted into a new disease management system 2000. While these advantages are described with reference to disease management system 2000 some or all of these advantages may be applied to disease management system 1101 or other systems and methods described herein.

In the illustrated example, the disease management system 2000 contains various modular components. For example, the disease management system 2000 may contain a controller module 2102, a sensor injector 2104, a pump 2112, a medication injector 2116, and a pouch assembly 2118. In the illustrated example, the disease management system 2000 may also contain components to facilitate the interaction of the modular components. For example, the disease management system 2000 contains an adhesive layer 2106, a base 2108, an electrical connector 2110, and a printed circuit board assembly (PCBA) 2114.

In the illustrated example, the base 2108 may be configured to couple the controller module 2102, sensor injector 2104, pump 2112, PCBA 2114, medication injector 2116, and pouch assembly 2118 on one side of the base 2108 and to couple the adhesive layer 2106 on the opposite side of the base 2108. The base 2108 is further configured to house the electrical connector 2110. In some examples, the base 2108 may include a bandage. For example, a base 2108 may be a flexible bandage configured to conform to an application site on the body of a patient. In the above example, the flexible bandage allows for potentially rigid components (for example, the sensor injector 2104 and the medication injector 2116) to be placed in a specific configuration, regardless of the varying size and shape of a patient to achieve the desired treatment effect. In some examples, an adhesive layer 2106 is configured to temporarily affix and/or couple the base 2108 to the skin of a patient. For example, an adhesive layer 2106 may comprise an acrylic polymer configured to hold and/or couple or partially couple a base 2108 on a skin site of the patient until removal of the base 2108 for the skin site of the patient is desired.

In the illustrated example, the modular components of a disease management system 2000 may be housed in self-contained modules. For example, a controller module 2102, a sensor injector 2104 and a pouch assembly 2118 may all be housed in self-contained modules. In this example, while the self-contained modules may be rigid, the flexibility of the base 2108 and the separation of the modules provides additional flexibility to ensure proper alignment on the patient.

In the illustrated example, the base 2108 has a dimensions L′, W′, and D′. L′ and W′ are of sufficient length such that all components of the disease management system 2000 can be situated on the base 2108. L′ and W′ also depend on the medication administration needs of the patient. For example, both a sensor injector 2104 and a medication injector 2116 may need access to specific injection sites on the patient. In this example, L′ and We are chosen so that the sensor injector 2104 and medication injector 2116 are properly aligned. D′ must be of sufficient length such that the base 2108 has sufficient support for the components of the disease management system 2000. In contrast, if D′ must also be sufficiently thin to allow a sensor injector 2104 and a medication injector 2116 to access the skin site of a patient. While these factors for L′, W′, and D′ are considerations in product design, a skilled artisan will note that many lengths of L′, W′, a D′ are possible without changing the invention.

In various implementations, reducing the profile of the sensor injector 2104 and/or the medication injector 2116 may be beneficial to a patient. For instance, as the disease management system 2000 may be applied to a patient (e.g., applied on the patient's abdomen or low back) reducing the profile of the sensor injector 2104 and/or the medication injector 2116 may reduce the overall thickness of the disease management system 2000, allowing for improved comfort and maneuverability of the patient.

In the illustrated example, one or more modules of the disease management system 2000 may be coupled to the base 2108. Additionally, one or more modules of the disease management system 2000 may be electrically coupled to other modules through at least one electrical connector 2110 associated with the base 2108. In the illustrated example, the sensor injector 2104, the controller module 2102, the PCBA 2114, and the medication injector 2116 are electrically coupled together by the electrical connector 2110. In some examples, a controller module 2102 may contain a controller and a battery such that the controller module 2102 sends (e.g., transmits) electrical power and electrical control signals through the electrical connector 2110. For example, the controller may include one or more hardware processors, PCBs ICs, ASICs, and/or the like. The controller may be configured to communicate with peripheral devices and/or components to support the accurate measurement of physiological parameters and blood analytes, such as patient pulse, temperature, and blood glucose, using detector electronics. The controller may subsequently calculate dose or receive a calculated dose value and administer medication, such as insulin, by actuation of an actuated pump. The controller may record device activity and transfer the recorded data to non-volatile secure memory space. At the end of the life of a device or system, the controller can be configured to lock operation, and create a data recovery module to permit authenticated access to the recorded data if needed.

In the illustrated example, the controller module 2102 may also include one or more communication components. A communication component can include, but is not limited to one or more radios configured to emit Bluetooth, cellular, Wi-Fi, or other wireless signals. In some examples, a communication component can include a port for a wired connection. Additionally, the controller module 2102 may include an NFC tag to facilitate in communicating with one or more hardware processors. The one or more communication components and NFC tag may be configured to communicate with the controller in order to send and/or receive information associated with the disease management system 2000. For example, a controller may communicate medication information and measured values through the one or more communication components to an external device. Additionally, the controller may receive instructions associated with measurement sampling rates, medication delivery, or other information associated with operation of the disease management system 2000 through the one or more communication components from one or more external devices.

In the illustrated example, one or more electrical connectors 2110 may be configured to electrically couple one or more components of the system 2000. For example, the one or more electrical connectors 2110 may be physically coupled to the base 2108. In some examples, electrical connectors 2110 may be embedded in the base 2108. For example, electrical connectors 2110 may be milled into base 2108. In another example, electrical connectors 2110 may be a printed circuit inlayed into base 2108. In another example, electrical connectors 2110 may be wires couple to the base 2108. However, other methods of coupling electrical connectors 2110 to base 2108 may be used. Components of the system 2000 may be oriented to couple to the base 2108 such that the one or more electrical connectors 2110 may electrically contact with one or more electrical components of other components of the system 2000. In some examples, the one or more electrical connectors 2110 may include a single electrical connector configured to travel along at least a portion of the length of the base 2108 to allow electrical contact between different portions (or components) of the system 2000, such as the controller module 2102, the PCBA 2114, the sensor injector 2104 and/or the medication injector 2116. The one or more electrical connectors 2110 may be configured to have a similar flexibility to that of the base 2108 such that when the base moves or flexes, the one or more electrical connectors also moves or flexes. In the illustrated example, the electrical connector 2110 is a flexible cable connector that has been embedded into milled out portions of the base 2108. However, other methods of electrical connection may be used such as the methods of electrical connections discussed above.

In the illustrated example, the disease management system 2000 may be applied to a patient. For example, the disease management system may be placed on an administration site on the skin of a patient that is typical for the administration of a medication to a patient (e.g., the lower back, the lower abdomen, the upper arm, or any other potential administration site). In this example, an adhesive layer 2016 holds and/or couples or partially couples the disease management system 2000 in place on the skin of a patient at the administration site. Once the disease management system 2000 is in place at the administration site, the patient, or another person, may trigger a medication injector 2116 to insert a cannula into the patient. For example, the patient, or another person, may physically interact with an interface to trigger the medication injector 2116 to insert the cannula into the patient. In another example, a patient, or another person, may cause a controller module 2102 to send an electrical signal via the electrical connector 2110 to trigger a medication injector 2116 to insert a cannula into the patient. The cannula may be coupled to a pump 2112. The pump 2112 may be configured to draw medication (such as insulin, glucagon, or other medication) from a pouch assembly 2118 such that the pump 2112 draws the medication from the pouch assembly 2118 through the cannula of the medication injector 2116 and into the patient. The pump 2112 may be electrically connected to the PCBA 2114 such that the pump 2112 may receive electrical signals provided by the electrical connector 2110 via the PCBA 2114. In some implementations, the PCBA 2114 may provide physical and electrical support for the pouch assembly 2118 and/or the pump 2112. For example, the PCBA 2114 may comprise a rigid structure and various electrical components, such as power filtering and/or converting circuitry.

In the illustrated example, a patient, or another person, may trigger a sensor injector 2104 to insert an analyte sensor into the patient. In some implementations, the analyte sensor may be an electrochemical analyte sensor, an optical sensor, or another sensor used to measure an analyte. For example, the patient, or another person, may physically interact with an interface to trigger the sensor injector 2104 to insert an analyte sensor into a patient. In another example, the patient, or another person, may cause a controller module 2102 to send an electrical signal via the electrical connector 2110 to trigger the sensor injector 2104 to insert an analyte sensor into the patient. The analyte sensor may be configured to provide readings of a patient's blood glucose levels (e.g., through a continuous glucose monitoring (CGM) system) or another blood analyte and to communicate those readings through an electrical signal via the electrical connector 2110 to the controller module 2102. For example, after an analyte sensor configured to provide readings of a patient's blood glucose level is inserted into the patient, the controller module 2102 may receive glucose readings from the analyte sensor via the sensor injector 2104. The controller module 2102 may use these glucose readings to control the pump 2112 to increase or decrease the medication drawn from the pouch assembly 2118 into the patient via the cannula inserted by medication injector 2116. By doing so, the controller module 2102 is able to stabilize the glucose levels in the patient to a target point.

FIG. 2G shows a block diagram illustrating an example process the controller module 2102 may use in administering medication to a patient. At block 2200, the controller module 2102 receives a reading from a sensor. For example, at block 2200 the controller module 2102 may receive a reading of a blood analyte from the sensor injector 2104 via the electrical connector 2110. At block 2202, the controller module 2102 determines whether the reading falls within a target threshold. For example, a processor in the controller module 2102 may compare the reading to a threshold stored in one or more nonvolatile memory units of the controller module 2102 or may compare the reading to a threshold received via a communication component. At block 2204, the controller module activates the pump to deliver a dose of medication to the patient following a determination that the reading was outside of the threshold. For example, if a reading falls outside of a threshold, the controller module 2102 may control the pump 2112 to send a dose of medication from the pouch assembly 2118 to the medication injector 2116 and into the patient via the injected cannula. In the example, the dose of medication can be a predetermined value stored in the controller module 2102 or communicated to the controller module 2102 via a communication component. In another example, the dose of medication may be continually administered until a reading that the patient levels fall within the threshold.

Referring again to FIGS. 2A-2E, some components of the disease management system 2000 may be reusable and some components may be disposable. Reusable components may be transferred from one disease management system to another. For example, the base 2108 and the electrical connectors 2110 may be configured to allow reusable components to decouple from the base 2108 and the electrical connectors 2110 of one disease management system 2000 and to couple to the base 2108 and electrical connectors 2110 of a different disease management system 2000. In one example, the controller module 2102 is reusable. In this example, when a patient needs to replace the disease management system 2000, the patient can remove the controller module 2102 from the disease management system 2000 and couple the controller module 2102 to the base 2108 of a second disease management system 2000. In this example, the second disease management system 2000 only contains a new sensor injector 2104, adhesive layer 2106, base 2108, electrical connector 2110, pump 2112, printed circuit board assembly (PCBA) 2114, medication injector 2116, and pouch assembly 2118, allowing the patient to save resources. Additionally, the controller module may store data pertinent to the patient's treatment, allowing for a more efficient transition to the second disease management system 2000.

In an alternate example, the disease management system 2000 may contain multiple medication injectors 2116, sensor injectors 2104 and/or pouch assemblies 2118. For example, a disease management system 2000 configured to stabilize the blood glucose levels of a patient may have a sensor injector 2104 configured to inject an analyte sensor to monitor the blood glucose levels of a patient, a first medication injector 2116 configured to inject insulin from a first pouch assembly 2118 in response to the patient's blood glucose level rising above a threshold value, and a second medication injector 2116 configured to inject glucagon from a second pouch assembly 2118 in response to the patient's blood glucose level falling below a different threshold value. In the above example the first and second medication injectors 2116 and pouch assemblies 2118 are described as being contained in the same disease management system 2000. A skilled artisan will understand that other configurations may accomplish a similar result such as multiple disease management systems 2000 configured to administer different medications, such as insulin, glucagon or other medications, either with established communication between individual systems or without.

In an alternate example, redundant disease management systems 2000 may be used. For example, a first disease management system 2000 may be implemented to stabilize the blood glucose levels of a patient and a second disease management system 2000 may be used at the same time and activated if a failure of the first disease management system 2000 occurs or to activate when the first disease management system 2000 requires replacement.

In an alternate example, no sensor injector 2104 is present on the disease management system 2000. In this example, the controller module 2102 controls the administration of medication through the medication injector 2116 based on one or more parameters other than sensor data from a sensor injector. For example, the controller module 2102 may control the administration of medication through the medication injector 2116 based on a set schedule, user input by a patient or other person, or other parameters used by skilled artisans in determining the proper administration of medication to a patient.

While the above examples describe the disease management system 2000 in connection with the control of a patient's blood glucose levels, a skilled artisan can appreciate that disease management system 2000 can be used to monitor and administer other medications as well. For example, the disease management system 2000 may be used to monitor patient blood and administer epinephrine to a patient. In another example, the disease management system 2000 may be used to monitor and deliver sedatives to a patient. While specific examples are described, a skilled artisan will appreciate that other medications may be used as well.

FIG. 2F illustrates an exploded view of the components of an example controller module 2102. In the illustrated example, the controller module 2102 contains a control unit 2502, a battery 2506, cutouts 2504, a compartment seal 2508, and an electrical contact seal 2510.

In the illustrated example, the control unit 2502 may contain any or all of the components as discussed with the controller module 2102 of FIGS. 2A-2E. The control unit may also contain computer microcontrollers, alarm indicators, a Bluetooth modem, and a 5G modem. The controller module 2102 may contain a battery 2506 to deliver power to the control unit 2502 and to the various modules of the disease management system 2000 via the electrical connector 2110. The controller module 2102 may contain cutouts 2504 to provide extra space to allow for high-profile components in the control unit 2502 and to allow the battery 2506 to expand due to changes in temperature.

In the illustrated example, the compartment seal 2508 and the electrical contact seal protect the control unit 2502 and the battery 2506 from water ingress. The compartment seal 2508 and the electrical contact seal 2510 may be created through an overmolded gasket. Alternatively, the compartment seal 2508 and the electrical contact seal 2510 may be created by a welding joint.

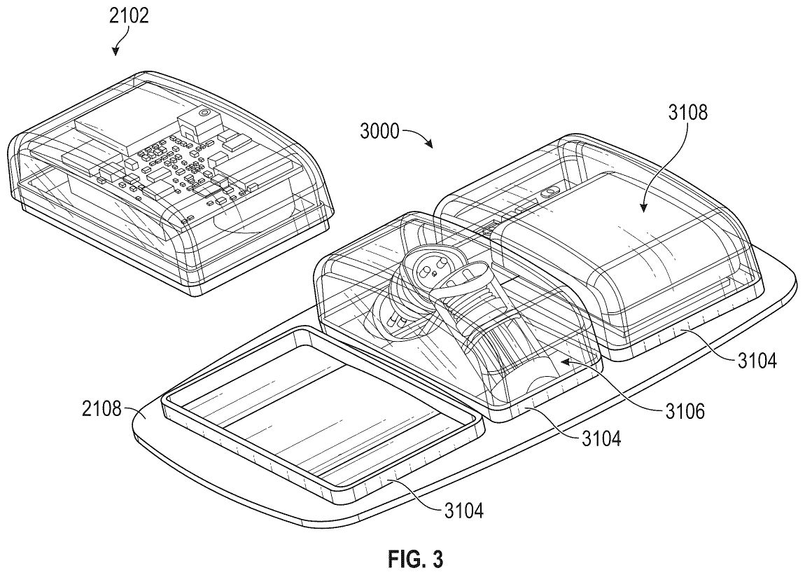

FIG. 3 illustrates a perspective view of an alternate example modular disease management system 3000. Modular disease management system 3000 may include all the components of disease management system 2000 with a reduced number of modules. For example, modular disease management system 3000 may include a controller module 2102, and a base 2108, similar to disease management system 2000, with a combination sensor and medication injector module 3106, and a pump module 3108 combining various modules of the disease management system 2000. Modular disease management system 3000 also include physical interfaces 3104. The base 2108 and the controller module 2102 may include all features described in FIG. 2A and FIG. 2F.

In the illustrated example, the physical interfaces 3104 couple the controller module 2102, the combination sensor and medication injector module 3106, and the pump module 3108 to the base 2108. Physical interfaces 3104 allow for the coupled elements to be easily connected and removed from the base 2108. For example, when a user wishes to replace the modular disease management system 3000, the user may remove the controller module 2102 from the old system and reuse the controller module 2102 with the new system. This reuse may be desirable to save cost or to keep consistent parameters that may be stored in the controller module 2102.

In the illustrated example, the pump module 3108 may include a reservoir pouch storing a substance to be administered to a user. When the reservoir is depleted, the user may replace the pump module 3108 without replacing the entire modular disease management system 3000.

In the illustrated example, the combination sensor and medication injector module 3106 may include a medication injector and a sensor injector such as the medication injector 2116 and the sensor injector 2104 described in FIGS. 2A-2E. In contrast to FIG. 2A, the combination sensor and medication injector module 3106 is placed centrally on the modular disease management system 3000. For example, the combination sensor and medication injector module 3106 may allow a sensor injector, for example sensor injector 2104, and a medication injector, for example medication injector 2116, to be placed in close proximity with each other. In this example, the close proximity may help facilitate proper alignment of both the medication injector and the sensor injector to an administration site on the patient.

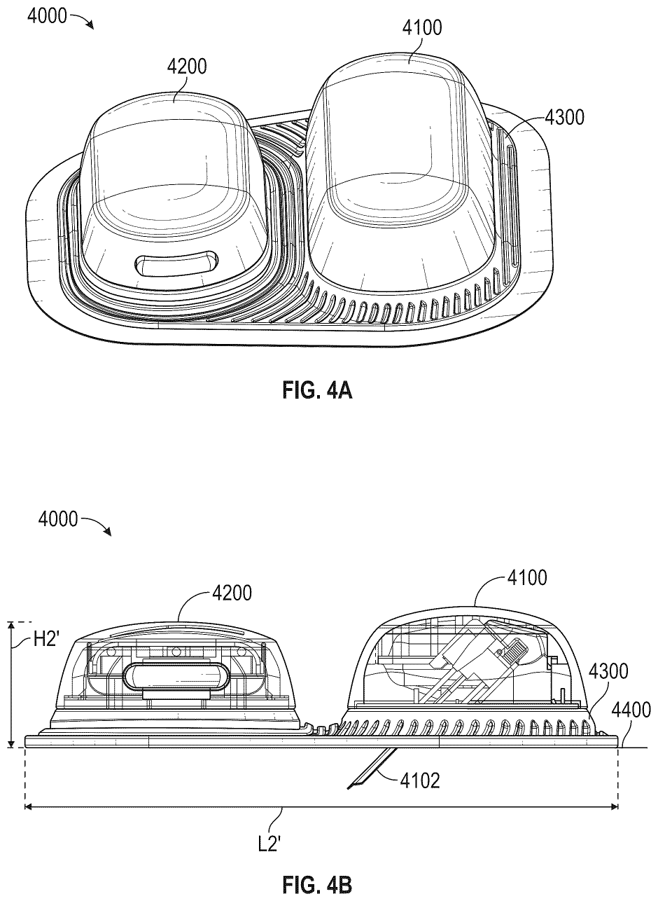

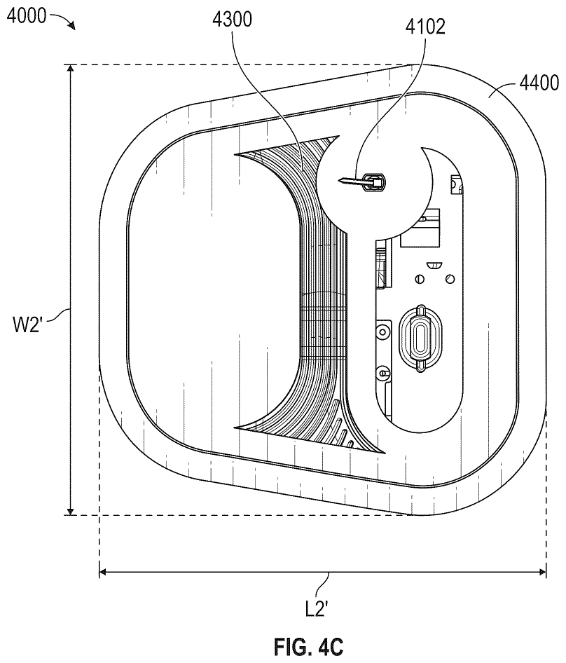

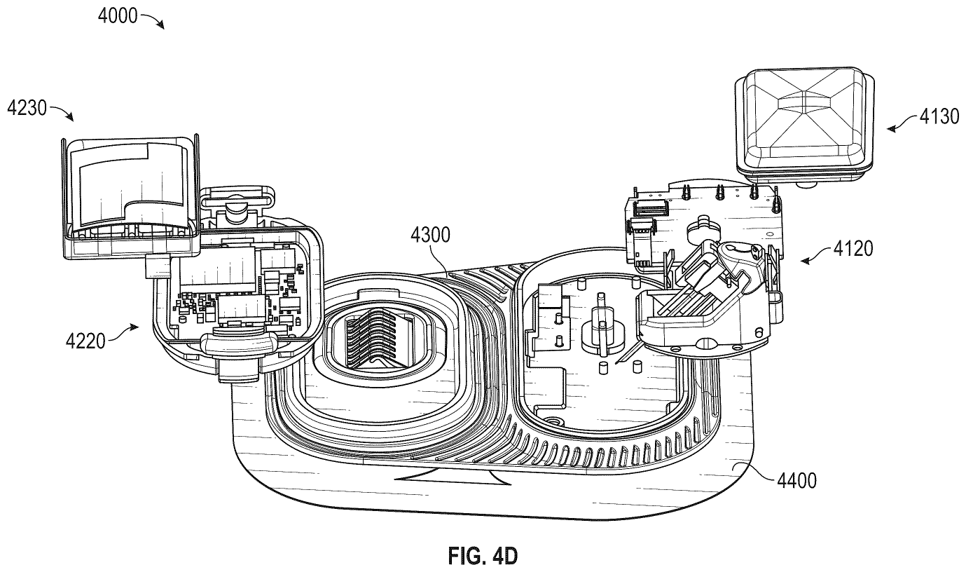

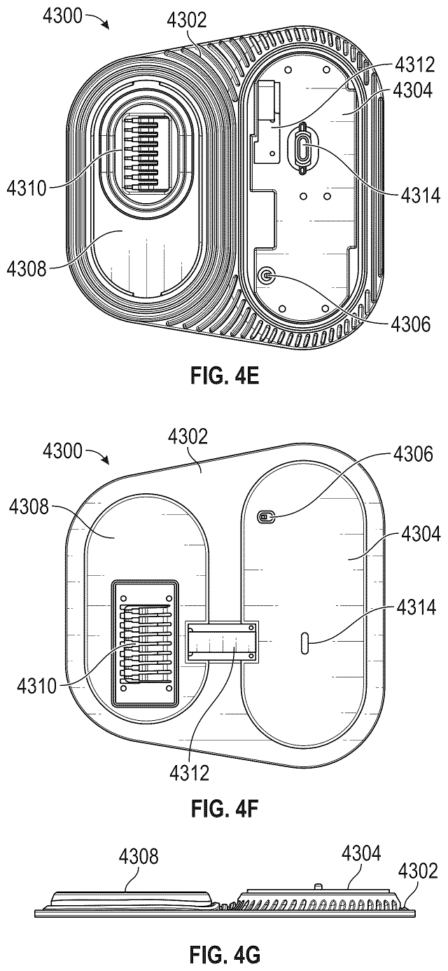

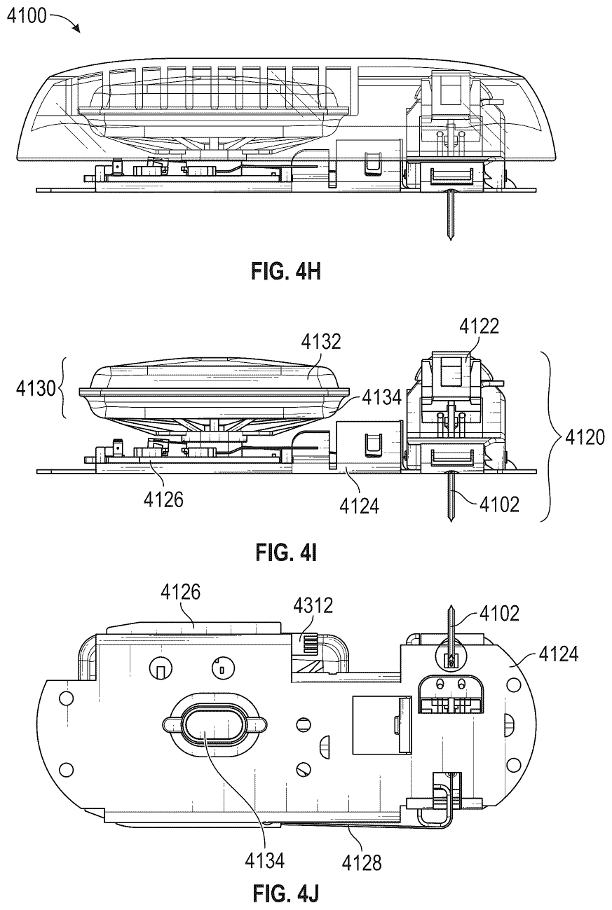

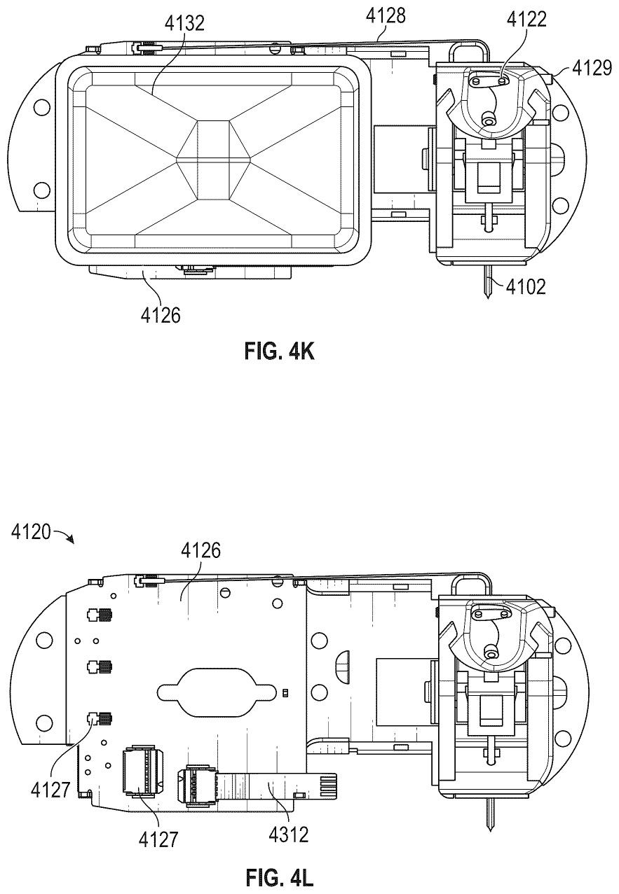

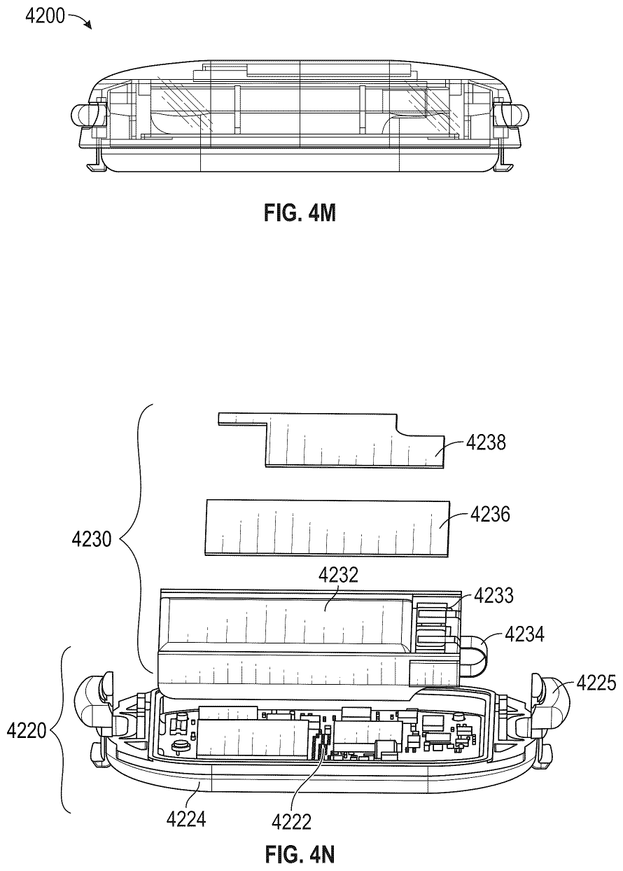

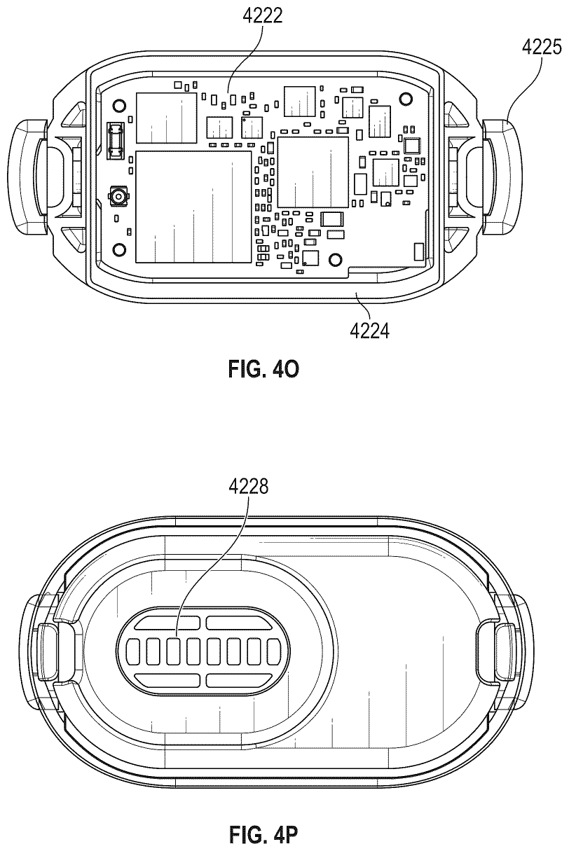

FIGS. 4A-4P illustrate example aspects of another embodiment of a disease management system 4000. In various implementations, disease management system 4000 may include some of, or all of, the components of disease management system 2000 and/or disease management system 3000. In some implementations, disease management system 4000 may include a reduced number of modules. In some implementations, disease management system 4000 may include a single needle insertion device. FIG. 4A illustrates a perspective view of the disease management system 4000. FIG. 4B illustrates a side view of the disease management system 4000. FIG. 4C illustrates a bottom view of the disease management system 4000.

As illustrated in FIGS. 4A-4C, the disease management system 4000 can include a disposable module 4100, a reusable module 4200, a base 4300, and an adhesive layer 4400. The disease management system 4000 may have dimensions L2′, H2′, and W2′. The value of dimensions L2′ and W2′ may allow the disease management system 4000 to be placed on a patient, for example, on a patient's lower back. The value of dimension H2′ may be important to a patient. For example, a reduction in the value of dimension H2′ may increase patient comfort, be more convenient for patients, and/or provide other benefits.

As will be described in more detail with respect to FIGS. 4E-4G, the base 4300 can include housing components for the reusable module 4200 and the disposable module 4100. The base 4300 can allow for a needle 4102 to be freely extended and retracted through the base 4300 (e.g., the needle 4102 may extend and retract through needle via 4306). In some implementations, the base 4300 can include rigid portions configured to provide structure to the disease management system 4000. In some implementations, the base 4300 can also include flexible portions, allowing the disease management system 4000 to contour along the skin of a patient. In some implementations, the base 4300 includes grooves to provide the flexible portions. The base 4300 may be attached to and/or include the adhesive layer 4400. The adhesive layer may be configured to temporality affix and/or couple or partially couple the base 4300 to the skin of a patient. For example, an adhesive layer 4400 may comprise an acrylic polymer configured to hold a base 4300 on a skin site of the patient until removal of the base 4300 for the skin site of the patient is desired.

FIG. 4D illustrates an exploded view of the disease management system 4000, according to various implementations. As is illustrated in FIG. 4D, the disposable module 4100 can include a pump and insertion assembly 4120 and a pouch assembly 4130. The reusable module 4200 can include a controller module 4220 and a battery module 4230. As described above, the reusable module 4200 may be removed (e.g., disconnected) from one disease management system 4000 and inserted into another disease management system 4000. As such, when the disease management system 4000 is replaced the reusable module 4200 may be reused, saving overall cost and allowing continuity of data between each disease management system 4000.

FIGS. 4E-4G illustrate views of the base 4300, according to various implementations. FIG. 4E illustrates a top view of the based 4300. FIG. 4F illustrates a bottom view of the based 4300. FIG. 4G illustrates a side view of the based 4300. The base 4300 can include a flexible portion 4302, a reusable module housing 4308, a disposable module housing 4304, a reusable module electrical connector 4310, an electrical connector 4312, a needle via 4306, and a pouch housing 4314.

In various implementations, the reusable module housing 4308 may be configured to physically couple the reusable module 4200 to the base 4300. The reusable module housing 4308 may include a sealed portion, such as the electrical contact seal 2510 described with respect to FIG. 2F. The reusable module electrical connector 4310 may be positioned within the reusable module housing 4308, such that when the reusable module 4200 is electrically coupled to the base 4300, the reusable module electrical connector 4310 electrically couples to the reusable module 4200.

In various implementations, the disposable module housing 4304 may be configured to physically couple the disposable module 4100 to the base 4300. The disposable module housing 4304 may provide a seal to prevent fluid, air, and/or other contaminants from entering the disposable module 4100. The needle via 4306 may be positioned in the disposable module housing 4304 beneath a needle insertion device, such as the needle insertion device 4122 described below, such that a needle may be freely inserted and removed from a patient through the base 4300. The pouch housing 4314 may be positioned in the 4304 beneath a pouch assembly, such as the pouch assembly 4130 described below. The pouch housing 4314 may physically couple to a pouch assembly to provide further support to the pouch assembly. In some implementations, pouch housing 4314 may include holes, tubes, and/or other passageways to connect a pump to a pouch assembly. In some implementations, the pouch housing 4314 includes access through the base 4300, such that the pouch assembly can be accessed from the bottom of the base 4300.

In some implementations, the reusable module housing 4308 and the disposable module housing 4304 may be rigid, or substantially rigid, to provide stability to the disease management system 4000. The flexible portion 4302 may be flexible to allow the disease management system 4000 to adapt to the contours of a patient's body and/or to allow increased range of movement for a patient with a disease management system 4000 affixed. In some implementations, the flexible portion 4302 may comprise a rigid, or semirigid material, with grooves to allow for flexibility. In other implementations, the flexible portion 4302 may be comprise a flexible material without the grooves illustrated in FIGS. 4E-4G.

The electrical connector 4312 can extend from the reusable module electrical connector 4310 to the disposable module 4100 such that electrical signals and/or power can be delivered from the reusable module 4200 to the disposable module 4100. The electrical connector 4312 can include any of the features of the electrical connector 2110 described above.

FIGS. 4H-4K illustrate a disposable module 4100, according to various implementations. FIG. 4H illustrates a side view of the disposable module 4100 with a cover. FIG. 4I illustrates a side view of the disposable module 4100 without a cover. FIG. 4J illustrates a bottom view of the disposable module 4100. FIG. 4K illustrates a top view of the disposable module 4100. FIG. 4L illustrates a top view of a pump and insertion assembly 4120 of the disposable module 4100.

As illustrated in FIGS. 4H-4K, the disposable module 4100 can include, but is not limited to, a pouch assembly 4130, with a medication pouch 4132 and a pouch structure 4134, and a pump and insertion assembly 4120, with a needle insertion device 4122 (also referred to as a “needle launcher”), needle 4102, pump and insertion base 4124, PCBA 4126, connectors 4127, engaging wire 4128, and cannula 4129.

The needle insertion device 4122 may be used to insert the cannula 4129 and/or and sensor into a patient using the needle 4102. In some implementations, the needle insertion device 4122 inserts the needle 4102 at an angle at the insertion site (also referred to as a “tissue site”) on a patient. For example, in some implementations the needle insertion device 4122 may insert the needle into a patient at approximately of 45 degree angle, at an angle less than a 45 degrees, or at an angle above 45 degrees. The engaging wire 4128 may be physically and/or electrically connected to the needle insertion device 4122 and may be configured to engage the needle insertion device 4122 in order to cause the needle insertion device 4122 to apply the needle into the patient. In some implementations, the engaging wire 4128 may be physically connected to a trigger mechanism, such as an actuator, retracting wire, trigger arm, and/or other trigger mechanism. In some implementations, the engaging wire 4128 makes up a portion of the trigger mechanism. For example, the engaging wire 4128 may be a retracting wire. In some instances, the engaging wire 4128 may retract in response to an electrical signal sent by the reusable module 4200 via the electrical connector 4312, the PCBA 4126, and/or other electrical connectors.

In some embodiments, the needle insertion device 4122 can be a medication injector, such as medication injector 2116 of FIGS. 2A-2F. In some embodiments, the needle insertion device 4122 can be a sensor injector, such as sensor injector 2104 of FIGS. 2A-2F. In various implementations, the needle insertion device 4122 can include a cannula 4129. One end of the cannula 4129 can be inserted into the patient using the needle insertion device 4122. The other end of the cannula 4129 can be mechanically coupled to a pump to draw medication form the pouch assembly 4130 and into the patient. In some implementations, the pouch assembly 4130 may include the features of the pouch assembly 2118 of FIGS. 2A-2F. The pouch assembly 4130 can include a medication pouch 4132 that stores one or more medications that can be administered to a patient via a pump and cannula 4129. The pouch assembly 4130 can include a pouch structure 4134 that can provide support and/or protection for the medication pouch 4132, interface with the base 4300, such as by the pouch housing 4314, facilitate the connection to a pump, and/or the like.

In various implementations, the PCBA 4126 may provide physical and electrical support for the medication pouch 4132 and/or pump and insertion assembly 4120. For example, the PCBA 4126 may comprise a rigid structure and various electrical components, such as power filtering and/or power converting circuitry. The PCBA 4126 may be electrically connected to the reusable module 4200 via the electrical connector 4312. For example, the PCBA 4126 may receive control signals and other electrical signals from the reusable module 4200 and convey them to the pump and insertion assembly 4120, such as to a pump, via the connectors 4127 and/or to the needle insertion device 4122 via the engaging wire 4128. The connectors 4127 may physically and/or electrically couple to a pump, such as pump 2112 illustrated in FIG. 2A. The connectors can include shield clip connectors, pin connectors, and/or other electrical and physical connectors. In various implementations, the pump and insertion base 4124 can provide a rigid structure for the pump and insertion assembly 4120 and/or the pouch assembly 4130. The pump and insertion base 4124 may include one or more fastener to physically couple the needle insertion device 4122 the PCBA 4126, the pouch structure 4134, a pump, and/or other component to the pump and insertion base 4124.

FIGS. 4M-4P illustrate a reusable module 4200, according to various implementations. FIG. 4M illustrates a side view of the reusable module 4200. FIG. 4N illustrates an exploded perspective view of the reusable module 4200. FIG. 4O illustrates a tip view of a controller module 4220 in a reusable module 4200. FIG. 4P illustrates a bottom view of the reusable module 4200.

As illustrated in FIGS. 4M-4P the reusable module 4200 can include a controller module 4220, with a bottom tray 4224, physical connectors 4225, a controller board 4222, and electrical contacts 4228, and a battery module 4230. The battery module can include a battery 4232, with a battery PCB 4233 and a battery connection 4234, a shield 4236, and an antenna 4238.

The reusable module 4200 may contain some, or all, of the features of the controller module 2102 described in FIGS. 2A-2F and 3. The reusable module 4200 can be inserted into the reusable module housing 4308 such that the physical connectors 4225 physically couple to the reusable module housing 4308 and the electrical contacts 4228 electrically couple to the reusable module electrical connector 4310. Electrical signals and/or power can be carried from the reusable module 4200 to the disposable module 4100 through the reusable module electrical connector 4310 and the electrical connector 4312. The reusable module reusable module 4200 can be removed from the base 4300 by releasing the physical connectors 4225 such that the reusable module 4200 is no longer physically coupled to the reusable module housing 4308. When the physical connectors are released, the electrical coupling between the electrical contacts 4228 and the reusable module electrical connector 4310. As such, one reusable module 4200 may be removed from one disease management system 4000 and place into another disease management system 4000. The bottom tray 4224 may provide protection and/or support for the reusable module 4200 such that the reusable module 4200 can be removed from, transported, and placed in a disease management system 4000 without or without substantially damaging the reusable module 4200.

The controller board 4222 may include one or more hardware processors, PCBs ICs, ASICs, and/or the like. The controller board 4222 may be configured to communicate with peripheral devices and/or components to support the accurate measurement of physiological parameters and blood analytes, such as patient pulse, temperature, and blood glucose, using detector electronics. The controller board 4222 may subsequently calculate dose or receive a calculated dose value and administer medication, such as insulin, by actuation of an actuated pump. The controller board 4222 may record device activity and transfer the recorded data to non-volatile secure memory space. At the end of the life of a device or system, the controller board 4222 can be configured to lock operation, and create a data recovery module to permit authenticated access to the recorded data if needed.

In the illustrated example, the controller board 4222 may also include one or more communication components, such as antenna 4238. The antenna 4238 can include, but is not limited to one or more radios configured to emit Bluetooth, cellular, Wi-Fi, or other wireless signals. In some examples, a controller board 4222 can include a port for a wired connection. Additionally, the controller board 4222 and/or the antenna 4238 may include an NFC tag to facilitate in communicating with one or more hardware processors. The antenna 4238 and NFC tag may be configured to communicate with the controller board 4222 in order to send and/or receive information associated with the disease management system 4000. For example, a controller board 4222 may communicate medication information and measured values through the one or more communication components to an external device. Additionally, the controller board 4222 may receive instructions associated with measurement sampling rates, medication delivery, or other information associated with operation of the disease management system 4000 through the antenna 4238 from one or more external devices.

The battery 4232 may store electrical power that is used by the controller board 4222, the antenna 4238, and/or the components of the disposable module 4100. The battery 4232 may include portions for one or more cells of the battery 4232 to expand, such as the cutouts 2504 illustrated in FIG. 2F. The battery 4232 can include a battery PCB 4233 to manage the charge and/or discharge of the battery. For example, the battery PCB 4233 can include various power circuitry, such as a power converting circuitry, to control the flow of power and the voltage levels output by the battery 4232. The battery connection 4234 can electrically and physically couple the battery 4232 to the controller board 4222. In some implementations, the battery 4232 is rechargeable. For example, the battery 4232 can include lead-acid batteries, nickel-cadmium batteries, nickel-metal hydride batteries, lithium-ion batteries, and/or other rechargeable batteries. In some implementations, the battery 4232 is not rechargeable. In some implementations, the battery 4232 can be recharged while inserted in a disease management system 4000 (e.g., through an external connector). In some implementations, the battery 4232 can be recharged by removing the reusable module 4200 and connecting it to a charger (e.g., via a cable connection, a power docking station, and/or other charging technique).

In some implementations, the shield 4236 provides protection and isolation for the antenna 4238 from the rest of the reusable module 4200. For example, shield 4236 may comprise a ferrite material to provide electromagnetic shielding for the antenna 4238.

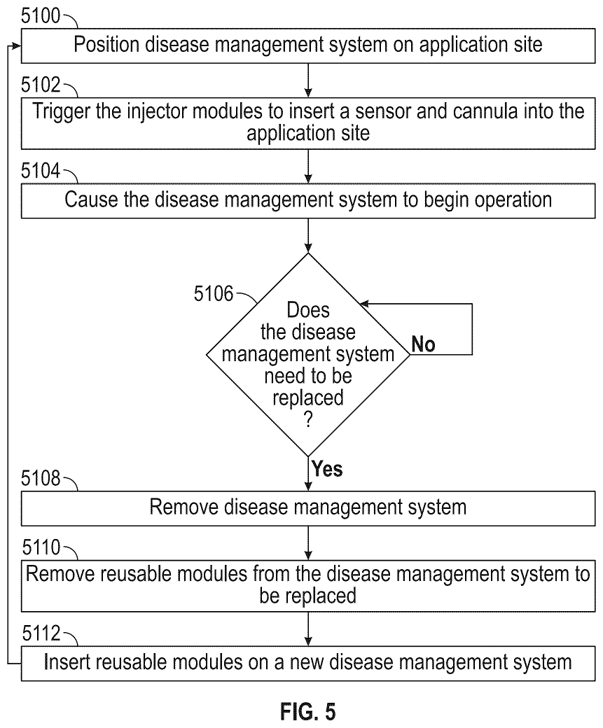

FIG. 5 illustrates an example process of a patient or other user of the disease management system 2000 of FIGS. 2A-2F, the modular disease management system of FIG. 3, and/or the disease management system 4000 of FIGS. 4A-4P. At block 5100 the user positions the disease management system on application site. For example, at block 5100 a user may affix and/or couple or partially couple the disease management system via an adhesive on the skin of a patient at the application site. At block 5102, the user triggers the injector modules to insert one or more sensors and cannulas into the patient at the application site. For example, the user may interact with a physical interface or cause a controller to send a signal to trigger one or more sensor injectors to inject an analyte sensor into the blood stream of a patient and to trigger one or more injectors to inject a medication delivering cannula into the blood stream of a patient. At block 5106, the user determines whether the disease management system is in need of replacement. For example, the user may determine that the disease management system must be replaced to avoid infection, that the medication reservoir has expired, or another reason that the disease management system must be replaced. When the disease management system must be replaced, at block 5108, the user removes the disease management system. For example, the user may peel of the disease management system, removing the adhesive from the patient. At block 5110, the user removes the reusable modules from the disease management system. For example, a user may remove a controller module, for example controller module 2102, from the disease management system. At block 5112, the user inserts the removed reusable modules into a new disease management system. For example, a user may insert a controller module, for example controller module 2102, that was taken from a previous disease management system, and insert the controller module onto a new disease management system. After block 5112, a user may restart the process to place the new disease management system on the patient.

### Example Guide Rail Automated Insertion Device

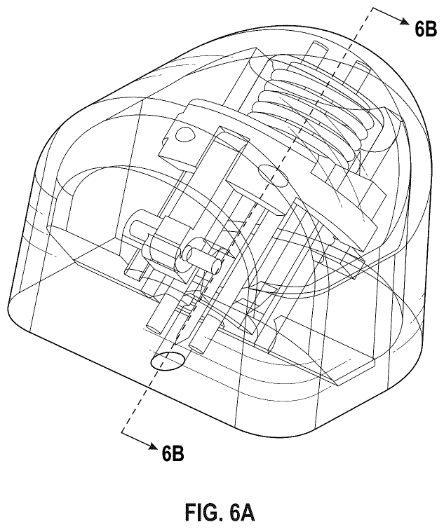

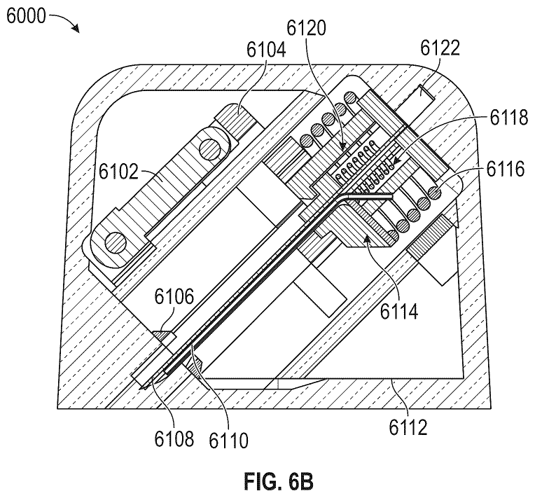

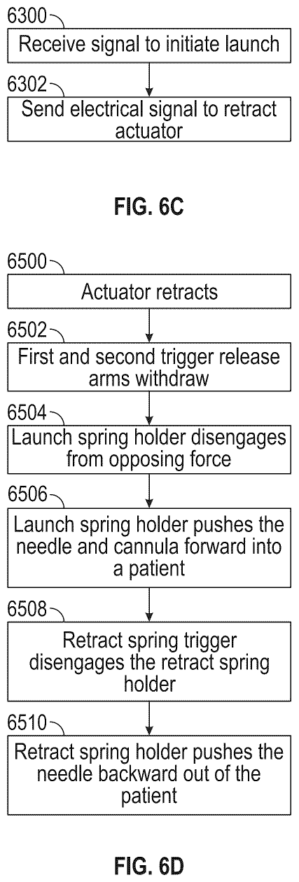

FIGS. 6A-6B illustrate an example automated insertion device 6000. In some examples, the automated insertion device 6000 may be part of a modular disease management system as described above. For example, the automated insertion device 6000 may be used as the sensor injector 2104 or medication injector 2116 of FIGS. 2A-2F or the needle insertion device 4122 of FIGS. 4A-4P and/or in the combination sensor and medication injector module 3106 of FIG. 3. As described above, reducing the overall profile of the automated insertion device 6000 can improve patient comfort and maneuverability. As such, automated insertion device 6000 advantageously provides easy insertion of a cannula or other device into a patient, while maintaining a low profile.

The automated insertion device 6000 may be used to insert a cannula or other device into an insertion site (also referred to as a “tissue site”) on the skin of a patient. In some implementations, the automated insertion device 6000 inserts the cannula at an angle at the insertion site on a patient. For example, the automated insertion device 6000 may insert the cannula into a patient at approximately a 45 degree angle, at an angle less than a 45 degrees, or at an angle above 45 degrees. The illustrated example contains a first release trigger 6102, a second release trigger 6104, a retract spring release 6106, a needle 6108, a cannula 6110, an enclosure 6112, a launch spring holder 6114, a launch spring 6116, a retract spring 6118, a retract spring holder 6120, and guide rails 6122.

Referring to FIG. 6A, the enclosure 6112 encases the other elements of the automated insertion device 6000. The enclosure 6112 also couples to the first release trigger 6102, creating a fixed hinge for the first release trigger 6102 to rotate inward. The second release trigger 6104 is coupled with the opposite end of the first release trigger 6102 such that when the first release trigger 6102 rotates inward the second release trigger 6104 rotates inward in the opposite direction.

Referring to FIG. 6B, the launch spring 6116 and the retract spring 6118 are preloaded and held in place by the launch spring holder 6114 and the retract spring holder 6120. The launch spring holder 6114 is in turn held in place by the second release trigger 6104. The needle 6108 is U-shaped, allowing the cannula 6110 to be positioned in the center of the needle 6108. For example, the needle 6108 may have a U-shaped cavity encompassing the cannula 6110. The needle 6108 and the cannula 6110 are positioned in the center of the retract spring 6118 which is in turn positioned in the center of the launch spring 6116.

The first release trigger 6102 is connected to an actuator (not shown) that is arranged to cause the first release trigger 6102 to rotate inward. For example, the actuator may be a nitinol muscle wire or an electrical actuator that is configured to retract in response to an electrical signal. When the actuator retracts, the first release trigger 6102 and the second release trigger 6104 rotate inward and the second release trigger 6104 withdraws from the launch spring holder 6114. Upon the second release trigger 6104 withdrawing from the launch spring holder 6114, the launch spring 6116 is released from the hold position, propelling the needle 6108 and the cannula 6110 forward along the guide rails 6122 and into a patient.

When the launch spring 6116 is fully extended (not shown) the retract spring holder 6120 interacts with the retract spring release 6106, forcing the retract spring holder 6120 upwards. When the retract spring holder 6120 is forced upwards, the retract spring 6118 is released from the hold position, propelling the needle 6108 backward along the guide rails 6122 out of the patient while the cannula 6110 remains inserted into a patient.

FIG. 6C illustrates an example processor a controller, for example a controller as discussed with reference to the controller module 2102 of FIGS. 2A-2E, may use to initiate the launch of the automated insertion device 6000. Beginning at block 6300, the controller receives a signal to initiate the launch of the automated insertion device. For example, the controller may receive a signal from a user interface or via a communication component to initiate the launch of an automated insertion device 6000. At block 6302, the controller sends a signal to retract the actuator. For example, the controller may send (e.g., transmit) electrical current to retract an actuator of the automated insertion device 6000. The actuator may be configured to retract in response to an electric signal. For example, the actuator may be a nitinol wire, or other composition, configured to retract in response to an electric current.

FIG. 6D illustrates an example process an automated insertion device, for example the automated insertion device 6000, may use to automatically insert a cannula into a patient. Beginning at block 6500, the actuator of the automated insertion device retracts. At block 6502, the first and second trigger release arms withdraw. For example, the actuator may be coupled to the second trigger release arm which is in turn coupled with the first trigger release arm. In the example, when the actuator retracts, the first release arm and the second release arm are withdrawn. At block 6504, the launch spring holder disengages from opposing force. For example, the first trigger release may apply an opposing force on the launch spring holder such that when the first trigger release withdraws the opposing force is removed. At block 6506, the launch spring holder pushes a needle and cannula forward into a patient. For example, the launch spring holder may interact with the needle and cannula such that the needle and cannula may be pushed forward by the needle holder. Furthering the example, the needle holder may also interface with a launch spring such that when the opposing force is removed, the needle holder is propelled forwarded and pushes the needle and cannula forward and into a patient. At block 6508, the retract spring trigger disengages the retract spring holder. For example, once the needle and cannula have been inserted into the patient, the retract spring trigger may physically disengage from the retract spring holder. At block 6510, the retract spring holder pushes the needle backward out of the patient. For example, the retract spring holder may interact with the needle such that the needle may be pushed backward by the retract spring holder. Furthering the example, the retract spring holder may also interface with a retract spring such that when the retract spring holder disengages with the retract spring holder, the retract spring pushes the retract spring holder backward and the needle out of the patient. While FIG. 6D describes a cannula, the process may be used to insert other items into a patient, for example an analyte sensor.

### Example Torsion Spring Automated Insertion Device

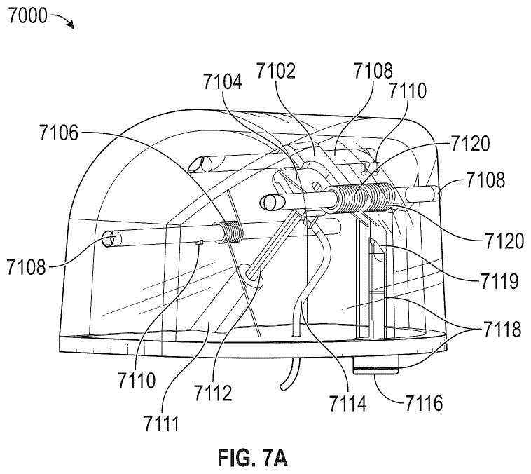

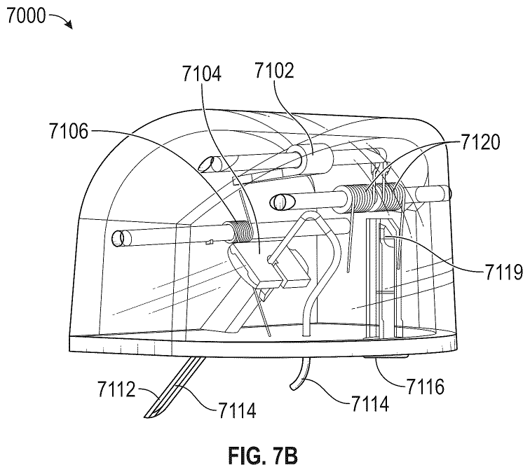

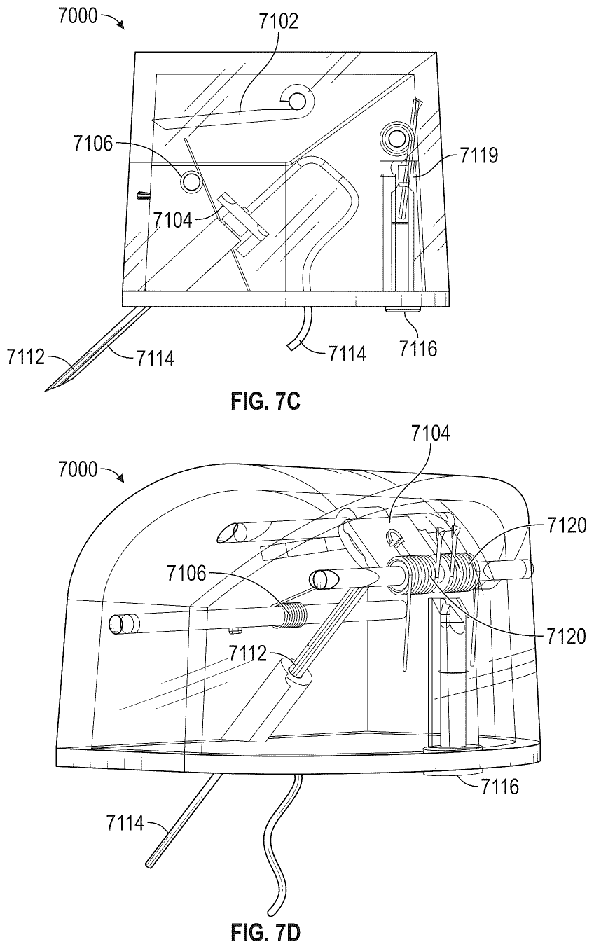

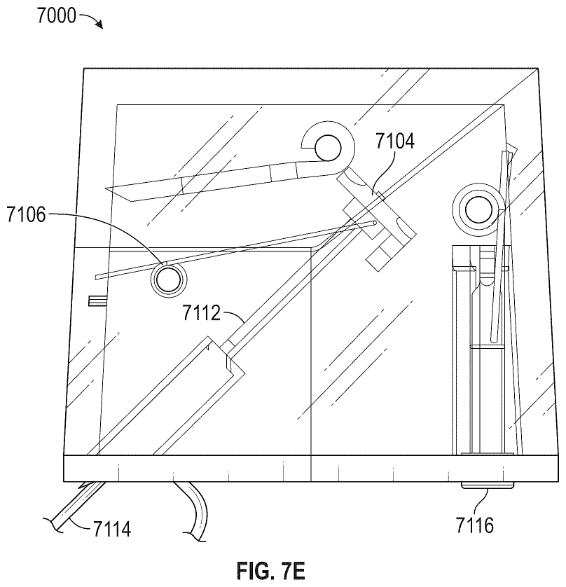

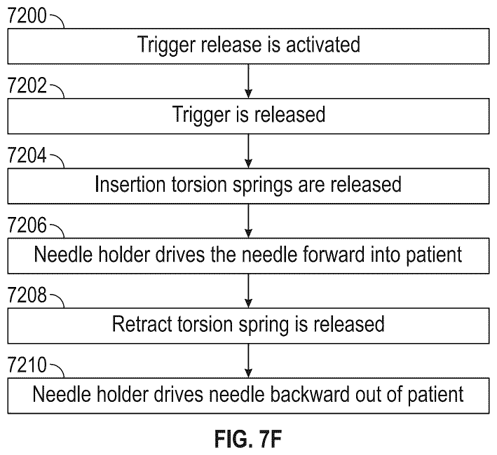

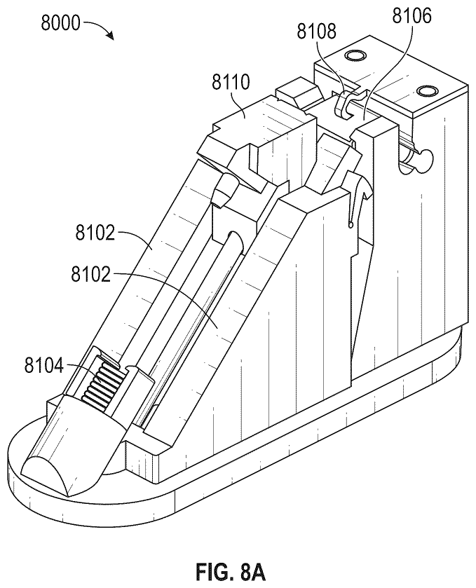

FIGS. 7A-7E illustrate an example torsion spring automated insertion device 7000. In some examples, the torsion spring automated insertion device 7000 may be part of a modular disease management system as described above. For example, the torsion spring automated insertion device 7000 may be used as the sensor injector 2104 or medication injector 2116 of FIGS. 2A-2F or the needle insertion device 4122 of FIGS. 4A-4P and/or in the combination sensor and medication injector module 3106 of FIG. 3. As described above, reducing the overall profile of the torsion spring automated insertion device 7000 can improve patient comfort and maneuverability. As such, torsion spring automated insertion device 7000 advantageously provides easy insertion of a cannula or other device into a patient, while maintaining a low profile.

The torsion spring automated insertion device 7000 may be used to insert a cannula or other device into an insertion site (also referred to as a “tissue site”) on the skin of a patient. In some implementations, the torsion spring automated insertion device 7000 inserts the cannula at an angle at the insertion site on a patient. For example, the torsion spring automated insertion device 7000 may insert the cannula into a patient at approximately a 45 degree angle, at an angle less than a 45 degrees, or at an angle above 45 degrees. The illustrated example includes a trigger 7102, a needle holder 7104, a retraction torsion spring 7106, rotating dowels 7108, spring engage pockets 7110, a guide rail 7111, a needle 7112, a cannula 7114, a trigger release 7116, O-ring seals 7118, a trigger release holder 7119, and insertion torsion springs 7120.

Referring to FIG. 7A, the torsion spring automated insertion device 7000 is shown in a pre-launch configuration. In the pre-launch configuration, the insertion torsion springs 7120 and the retraction torsion spring 7106 are at max spring tension. The insertion torsion springs 7120 and the retraction torsion spring 7106 are situated on the rotating dowels 7108 so that the insertion torsion springs 7120 and the retraction torsion spring 7106 rotate the rotating dowels 7108 as the spring tension is released. The insertion torsion springs 7120 are held at max spring tension by the trigger 7102. The trigger 7102 is in turn held in place from the opposing force of the insertion torsion springs 7120 by the trigger release holder 7119. One end of the insertion torsion springs 7120 engages with the needle holder 7104 so that the needle holder 7104 is driven forward by the insertion torsion springs 7120 as they rotate about the rotating dowels 7108. The retraction torsion spring 7106 is held at max spring tension by engaging with the guide rail 7111. The opposite end of the insertion torsion springs 7120 and one end of the retraction torsion spring 7106 are situated in the spring engage pockets 7110. The spring engage pockets 7110 provide the resistance for the insertion torsion springs 7120 and the retraction torsion spring 7106 are at max spring tension to rotate along the rotating dowels 7108 in the desired direction upon the release of the spring tension. O-ring seals 7118 provide a physical seal to the torsion spring automated insertion device 7000, preventing the ingress of fluids.

Referring to FIGS. 7B-7C, the torsion spring automated insertion device 7000 is shown in a launching configuration. In the launching configuration, the trigger release 7116 has been engaged, releasing the trigger 7102 from the trigger release holder 7119. Upon this release, the insertion torsion springs 7120 rotate about the rotating dowels 7108, driving the needle holder 7104 forward. The needle holder 7104 is coupled with the needle 7112 and the cannula 7114 so that the needle 7112 and the cannula 7114 are also driven forward along the guide rail 7111 and inserted into a patient. At maximum insertion, the needle holder 7104 disengages the retraction torsion spring 7106 from the guide rail 7111.

Referring to FIGS. 7D-7E, the torsion spring automated insertion device 7000 is shown in a retracting configuration. In the retracting configuration, the retraction torsion spring 7106 has been disengaged from the guide rail 7111, allowing the retraction torsion spring 7106 to rotate along the rotating dowel 7108 in the opposite direction as the insertion torsion springs 7120 had rotated as described above. While the retraction torsion spring 7106 rotates, the cannula 7114 is released from the needle holder 7104 and the retraction torsion spring 7106 drives the needle holder 7104 backwards, retracting the needle 7112 backwards along the guide rail 7111. When the needle 7112 is fully retracted, only the cannula 7114 remains inserted into the patient. Additionally, when the needle 7112 is fully retracted the insertion torsion springs 7120 and the retraction torsion spring 7106 have no tension.

FIG. 7F illustrates an example process a torsion spring automated insertion device, for example the torsion spring automated insertion device 7000, may use to automatically insert a cannula into a patient. Beginning at block 7200, the trigger release is activated. For example, trigger release 7116 may be depressed inward. At block 7202, the trigger is released. For example, the trigger release 7116 may disengage the trigger 7102 from the trigger release holder 7119. At block 7204, the insertion torsion springs are released. For example, the insertion torsion springs 7120 may be held in place by the opposing force of the trigger 7102 such that when the trigger is released, the insertion torsion springs 7120 are also released. At block 7206, the needle holder drives the needle forward into a patient. For example, a needle holder 7104 may be coupled with a needle 7112 such that when the needle holder 7104 is driven forward along a guide rail 7111 a needle is driven by the needle holder 7104 into a patient. At block 7208, a retract torsion spring is released. For example, when the needle holder 7104 is driven forward along the guide rail 7111, it may release a retraction torsion spring 7106 from a hold position. At block 7210, the needle holder drives needle backward out of the patient. For example, once the retraction torsion spring 7106 is released from the hold position the retraction torsion spring 7106 may apply a force on the needle holder 7104 such that the needle holder drives the needle 7112 backward along the guide rail 7111 and out of the patient. The process described in FIG. 7F may be used to automatically insert a device into a patient. For example, the process described in FIG. 7F may be used to automatically insert a cannula into a patient. In another example, the process described in FIG. 7F may be used to insert an analyte sensor or any other insertable device used for patient monitoring into a patient.

### Example Side Rail Automated Insertion Device

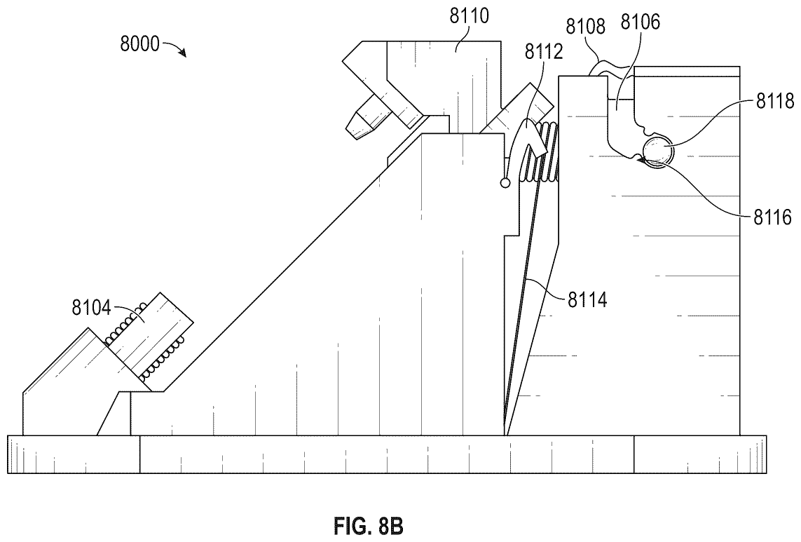

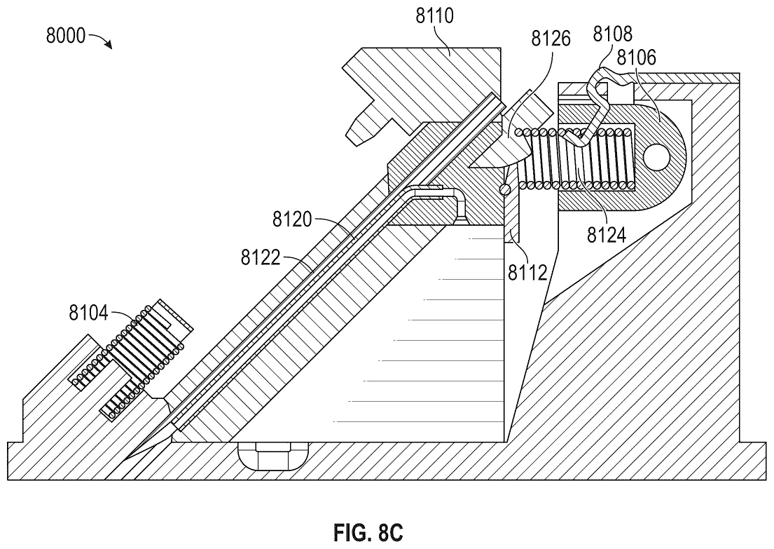

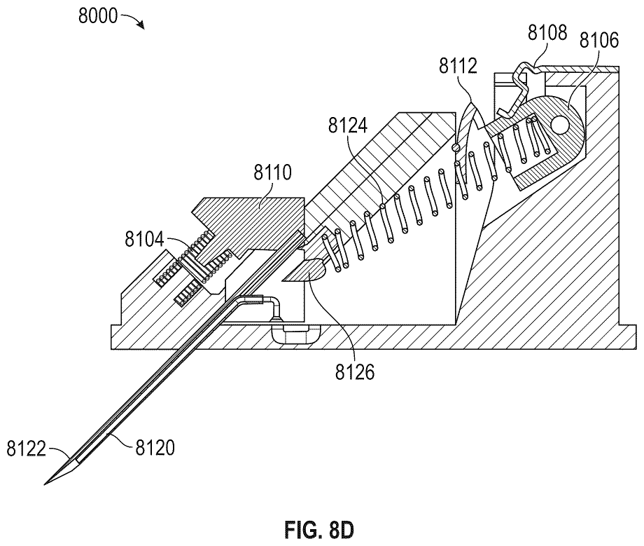

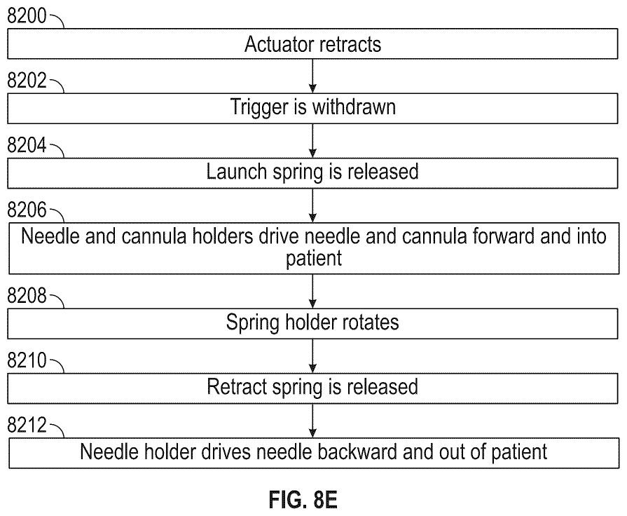

FIGS. 8A-8D illustrate an example side rail automated insertion device 8000. In some examples, the side rail automated insertion device 8000 may be part of a modular disease management system as described above. For example, the side rail automated insertion device 8000 may be used as the sensor injector 2104 or medication injector 2116 of FIGS. 2A-2F or the needle insertion device 4122 of FIGS. 4A-4P and/or in the combination sensor and medication injector module 3106 of FIG. 3. As described above, reducing the overall profile of the side rail automated insertion device 8000 can improve patient comfort and maneuverability. As such, side rail automated insertion device 8000 advantageously provides easy insertion of a cannula or other device into a patient, while maintaining a low profile.

The side rail automated insertion device 8000 may be used to insert a cannula or other device into an insertion site (also referred to as a “tissue site”) on the skin of a patient. In some implementations, the side rail automated insertion device 8000 inserts the cannula at an angle at the insertion site on a patient. For example, the side rail automated insertion device 8000 may insert the cannula into a patient at approximately a 45 degree angle, at an angle less than a 45 degrees, or at an angle above 45 degrees. The illustrated example includes side guide rails 8102, a retraction spring 8104, a spring holder 8106, a pressure spring 8108, a needle holder 8110, a trigger 8112, an actuator 8114, a rotation pin lock 8116, a rotation pin 8118, a cannula 8120, a needle 8122, a launch spring 8124, and a cannula holder 8126.

Referring to FIG. 8A-8C, the side rail automated insertion device 8000 is shown in a pre-launch configuration. In the pre-launch configuration, the retraction spring 8104 and the launch spring 8124 are in a hold position with the springs at max tension. The needle holder 8110 and the cannula holder 8126 are situated between the side guide rails 8102, restricting the movement of the needle holder 8110 and the cannula holder 8126 to forward and backward along the side guide rails 8102. The needle holder 8110 and the cannula holder 8126 are engaged with the launch spring so that the launch spring 8124 can push the needle holder 8110 and the cannula holder 8126 forward along the side guide rails 8102. The launch spring 8124 is held in the hold position by the trigger 8112. The launch spring 8124 is attached on one end to the spring holder 8106 to allow the launch spring 8124 to apply force on the needle holder 8110 and the cannula holder 8126. The spring holder 8106 is held in place by the rotation pin 8118, allowing the spring holder 8106 to rotate about the rotation pin 8118. In the pre-launch configuration, the spring holder 8106 is held in place from rotating by the rotation pin lock 8116. The pressure spring 8108 applies a force on the spring holder 8106 so that the spring holder 8106 rotates to align with the side guide rails 8102 once the rotation pin lock 8116 has been released. The needle 8122 is coupled with the needle holder 8110 and the cannula 8120 is coupled with the cannula holder 8126 such that when the needle holder 8110 and the cannula holder 8126 move along the side guide rails 8102 the needle 8122 and the cannula 8120 move as well. The actuator 8114 is connected to the trigger 8112 so that when the actuator 8114 is engaged, the trigger 8112 releases the launch spring 8124 from the hold position and the rotation pin lock 8116. The actuator 8114 can be physically or electrically activated. For example, the actuator 8114 can be a physical wire, and nitinol muscle wire, or other electrical actuator.

Referring to FIG. 8D, the side rail automated insertion device 8000 is shown in a launching configuration. The trigger 8112 has been released and the launch spring 8124 has pushed the needle holder 8110 and the cannula holder 8126 forward so that the needle 8122 and the cannula 8120 have been inserted into a patient. The pressure spring 8108 continues to apply pressure on the spring holder 8106 so that launch spring 8124 aligns with side guide rails 8102, thereby disengaging the needle holder 8110 from the launch spring 8124. Upon the needle 8122 and the cannula 8120 being fully inserted into the patient, the needle holder 8110 engages with the retraction spring 8104, forcing the needle holder 8110 backwards along the side guide rails 8102 and retracting the needle 8122 from the patient.

FIG. 8E illustrates an example process a side rail automated insertion device, for example the side rail automated insertion device 8000, may use to automatically insert a cannula into a patient. Beginning at block 8200, the actuator retracts. For example, actuator 8114 may retract. At block 8202, the trigger is withdrawn. For example, when the actuator 8114 retracts, the actuator 8114 may withdraw the trigger 8112. At block 8204, the launch spring is released. For example, the launch spring 8124 may be held in place by the opposing force of the trigger 8112 such that when the trigger is withdrawn, the launch spring 8124 is released. At block 8206, the needle holder and the cannula holder drive the needle and the cannula forward into a patient. For example, a needle holder 8110 may be coupled with a needle 8122 such that when the needle holder 8110 is driven forward along the side guide rails 8102, the needle 8122 is driven by the needle holder 8110 into a patient. Furthering the example, a cannula holder 8126 may be couple with a cannula 8120 such that when the cannula holder 8126 is driven forward along the side guide rails 8102, the cannula 8120 is driven by the cannula holder 8126 into the patient. In the example, the needle holder 8110 and the cannula holder 8126 are coupled to the launch spring 8124 such that when the launch spring 8124 is released the needle holder 8110 and the cannula holder 8126 are driven forward and the needle 8122 and the cannula 8120 are inserted into the patient. At block 8208, the spring holder rotates. For example, while the launch spring 8124 is driving the needle holder 8110 and the cannula holder 8126 forward, the rotation pin lock 8116 is released and the pressure spring 8108 applies a force on the spring holder 8106 such that the spring holder 8106 rotates along the rotation pin 8118. At block 8210, the retract spring is released. For example, when the spring holder 8106 rotates, the launch spring 8124 holds the cannula holder 8126 in a locked position beneath a return path of the needle holder 8110 on the side guide rails 8102 and the needle holder 8110 engages with the retraction spring 8104 such that the retraction spring 8104 is released. At block 8212 the needle holder drives the needle backward out of the patient. For example, when the retraction spring 8104 is released, the retraction spring 8104 applies a force on the needle holder 8110 such that the needle holder 8110 is driven backward along the side guide rails 8102 and the needle 8122 is withdrawn from the patient.

While FIGS. 8A-8E refer to the insertion of a cannula, it can be appreciated that other devices may be inserted with the side rail automated insertion device 8000. For example, an analyte sensor or other device can be used in place of the cannula.

### Example Single Rail Automated Insertion Device

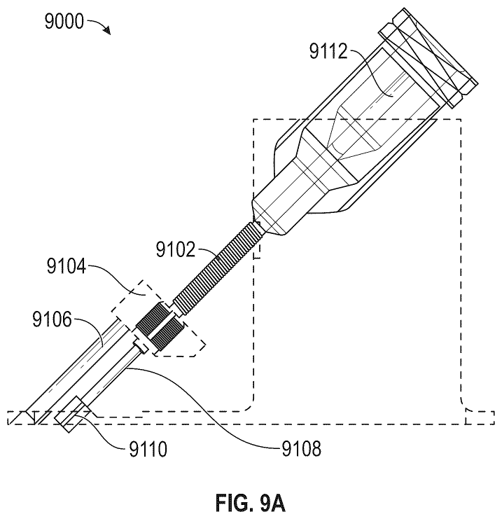

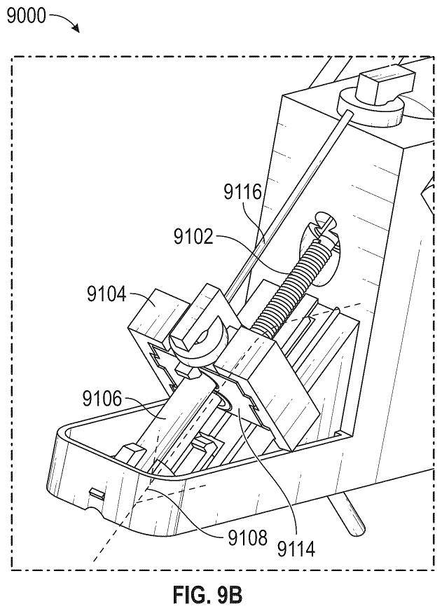

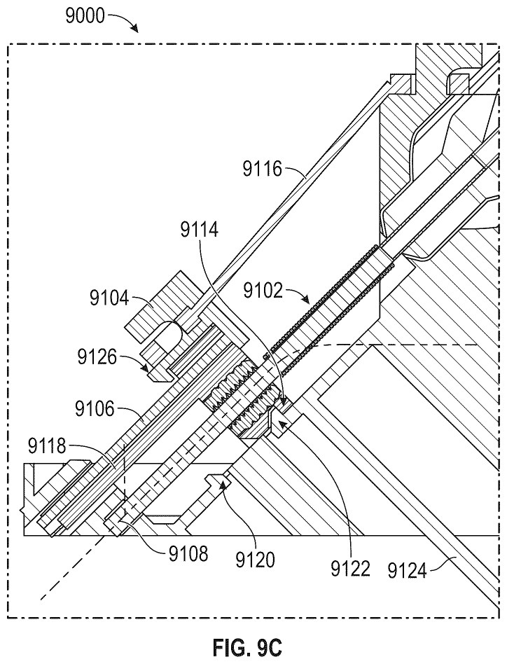

FIGS. 9A-9C illustrate an example single rail automated insertion device 9000. In some examples, the single rail automated insertion device 9000 may be part of a modular disease management system as described above. For example, the single rail automated insertion device 9000 may be used as the sensor injector 2104 or medication injector 2116 of FIGS. 2A-2F or the needle insertion device 4122 of FIGS. 4A-4P and/or in the combination sensor and medication injector module 3106 of FIG. 3. As described above, reducing the overall profile of the single rail automated insertion device 9000 can improve patient comfort and maneuverability. As such, single rail automated insertion device 9000 advantageously provides easy insertion of a cannula or other device into a patient, while maintaining a low profile.

The single rail automated insertion device 9000 may be used to insert a cannula or other device into an insertion site (also referred to as a “tissue site”) on the skin of a patient. In some implementations, the single rail automated insertion device 9000 inserts the cannula at an angle at the insertion site on a patient. For example, the single rail automated insertion device 9000 may insert the cannula into a patient at approximately a 45 degree angle, at an angle less than a 45 degrees, or at an angle above 45 degrees. The illustrated example includes a launch spring 9102, a needle carrier 9104, a needle 9106, a guide rail 9108, a guide rail anchor 9110, a spring anchor 9112, a cannula carrier 9114, a retract spring 9116, a cannula 9118, an insertion latch 9120, a trigger 9122, an actuator 9124 and a needle release 9126.

The single rail automated insertion device 9000 has a single guide rail 9108, thereby reducing the friction of the single rail automated insertion device 9000. The guide rail 9108 may be made of a guide wire. The guide rail anchor 9110 fixes the guide rail 9108 in place. For example, if guide rail 9108 is made of guide wire, the guide rail anchor 9110 allows the guide wire to maintain tension. The guide rail 9108 is coupled with the needle carrier 9104 and the cannula carrier 9114 so to restrict the movement of the needle carrier 9104 and the cannula carrier 9114 along a single axis. The needle carrier 9104 is coupled to the needle 9106 and the cannula carrier 9114 is couple to the cannula 9118 so that the needle 9106 and the cannula 9118 move along the same axis as the needle carrier 9104 and the cannula carrier 9114. The cannula carrier 9114 is coupled with the launch spring 9102 such that the launch spring 9102 moves the cannula carrier 9114 as the launch spring 9102 releases tension. The needle carrier 9104 is coupled with the retract spring 9116 such that the retract spring 9116 moves the needle carrier 9104 as the retract spring 9116 releases tension. The retract spring 9116 may comprise, for example, an elastic band or a compression spring. The needle carrier 9104 and the cannula carrier 9114 are couple together by the needle release 9126. As such, the needle carrier 9104 and the cannula carrier 9114 move together until the needle release 9126 is disengaged from the cannula carrier 9114.

When the single rail automated insertion device 9000 is in a pre-launch configuration, launch spring 9102 is at max tension, which exceeds the tension of the retract spring 9116. The cannula carrier 9114 is held in place against the force of the launch spring 9102 by the trigger 9122. The actuator 9124 is connected with the trigger 9122 as to cause the trigger 9122 to release when the actuator 9124 is retracted. For example, the actuator 9124 may be a nitinol muscle wire or an electrical wire configured to retract at an electrical signal, or a physical wire configured to receive a withdrawing force. When the actuator 9124 withdraws, the trigger 9122 is released, removing the opposing force on the cannula carrier 9114 and causing the launch spring 9102 to move the cannula carrier 9114 and the needle carrier 9104 along the guide rail 9108, causing the needle 9106 and the cannula 9118 to be inserted into a patient. As the tension in the launch spring 9102 decreases, the tension in the retract spring 9116 increases.

When the needle 9106 and the cannula 9118 have been inserted into the patient, the cannula carrier 9114 engages with the insertion latch 9120 holding the cannula carrier 9114 in a fixed position and needle release 9126 is disengaged from the cannula carrier 9114. The needle carrier 9104 is retracted by the retract spring 9116 removing the needle 9106 from the patient while the cannula 9118 remains inserted.

In an alternative embodiment, the needle 9106 and cannula 9118 as shown in FIGS. 9A-9C are replaced by a trocar system. The trocar system may include a trocar at the center of the cannula 9118 with the needle 9106 removed. The trocar system may allow for a reduced would size in the patient. Further, the trocar and cannula 9118 may be bent further reducing the overall profile of the single rail automated insertion device 9000. In this alternative embodiment, the trocar creates an insertion point for the cannula 9118 as the launch spring 9102 moves the cannula carrier 9114 along the guide rail 9108.

### Example Single Trigger Arm Automated Insertion Devices

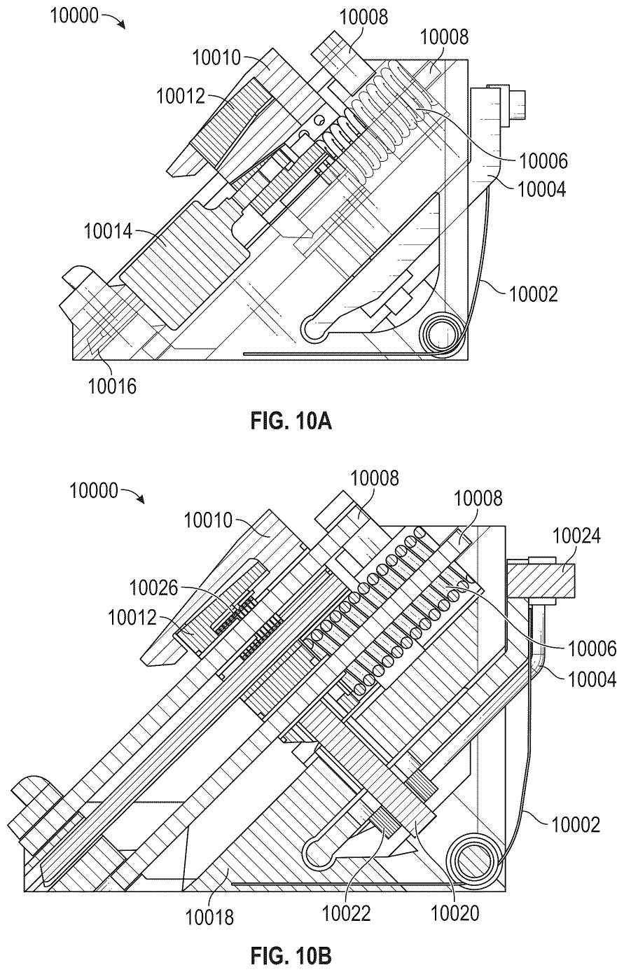

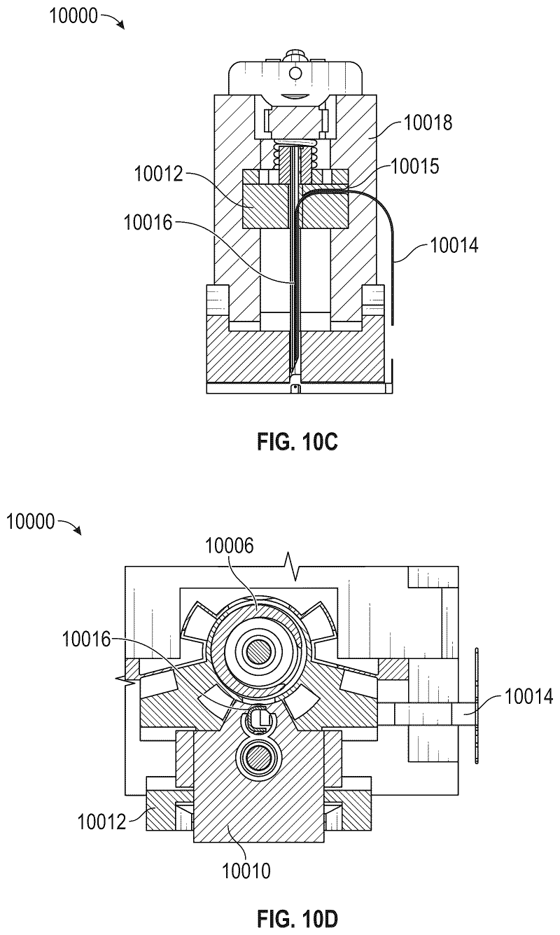

FIGS. 10A-10D illustrate an example automated insertion device 10000. In some implementations, the automated insertion device 10000 may be part of the modular disease management system as described above. For example, the automated insertion device 10000 may be used as the sensor injector 2104 of FIGS. 2A-2F or the needle insertion device 4122 of FIGS. 4A-4P and/or in the combination sensor and medication injector module 3106 of FIG. 3. As described above, reducing the overall profile of the automated insertion device 10000 can improve patient comfort and maneuverability. As such, automated insertion device 10000 advantageously provides easy insertion of a sensor or other device into a patient, while maintaining a low profile.

The automated insertion device 10000 may be used to insert a sensor or other device, such as the sensor 10014, into an insertion site (also referred to as a “tissue site”) on the skin of a patient. In some implementations, the automated insertion device 10000 inserts the sensor at an angle at the insertion site on the patient. For example, the automated insertion device 10000 may insert the sensor into the patient at approximately a 45 degree angle, at an angle less than a 45 degrees, or at an angle above 45 degrees. The illustrated example contains a trigger arm 10004, a retracting wire 10002, a launch spring 10006, guide rails 10008, a needle carrier 10010, a sensor carrier 10012, a sensor 10014, a sensor insertion opening 10015, a needle 10016, a body 10018, a trigger pin 10020, a stopper 10022, a roller 10024, and a retract spring 10026.

In some implementation, the sensor 10014 may be an analyte sensor, such a continuous glucose monitoring (CGM) sensor, or another sensor configured to make a reading a the insertion site on the skin of the patient. The automated insertion device 10000 may insert the sensor into the insertion site via the needle 10016. In various implementations, the needle 10016 can carry sensor 10014 into a patient. Then The automated insertion device 10000 may remove the needle 10016, leaving the sensor 10014 in the insertion site.

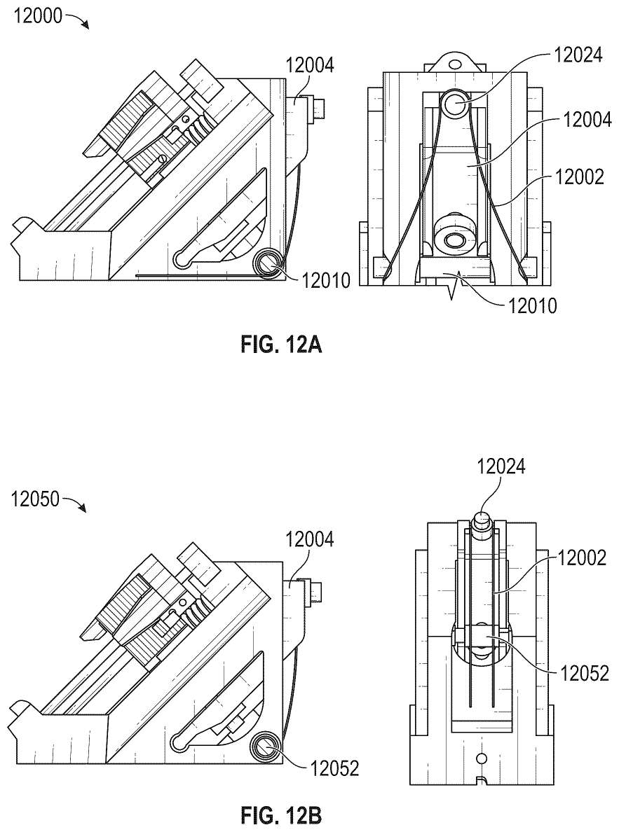

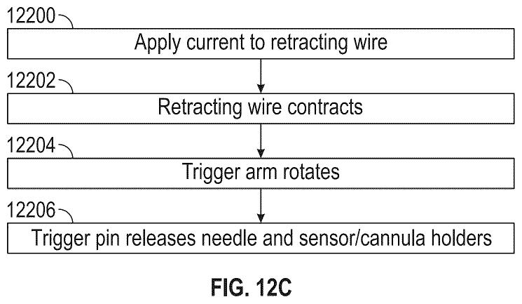

Referring to FIG. 10A, the body 10018 encases and provide structural support for the other elements of the automated insertion device 10000. In some implementations, the body 10018 also can couple to the retracting wire such that the retracting wire 10002 is anchored to the body 10018. In other implementations, at least one end of the retracting wire 10002 is coupled to a trigger mechanism that can apply a physical force and/or electrical current to the retracting wire 10002. In some implementations, the retracting wire 10002 may comprise a material with shape memory, such as nitinol, that forms a set state when exposed to electrical current. For example, the retracting wire 10002 may contract when an electrical current is applied to the retracting wire 10002. In other implementations, a physical force can be applied to the wire 10002 rather than electrical current. As will be described in further detail with respect to FIGS. 12A and 12B, the retracting wire 10002 be engaged about the roller 10024 such that the retracting wire 10002 may apply a force to the roller 10024 when the retracting wire 10002 retracts and/or transfer the physical force applied to the retracting wire 10002 to the roller 10024.

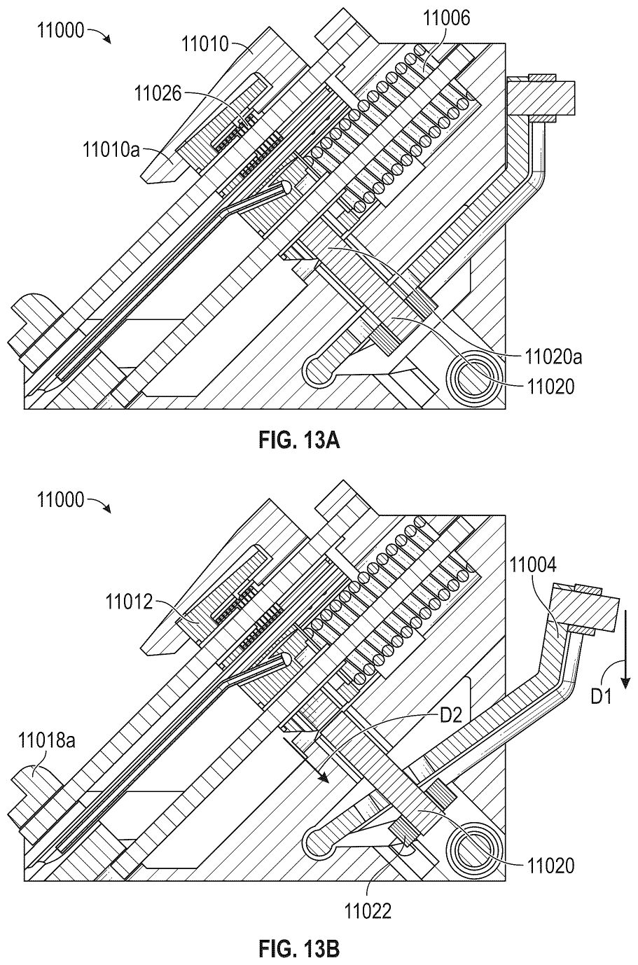

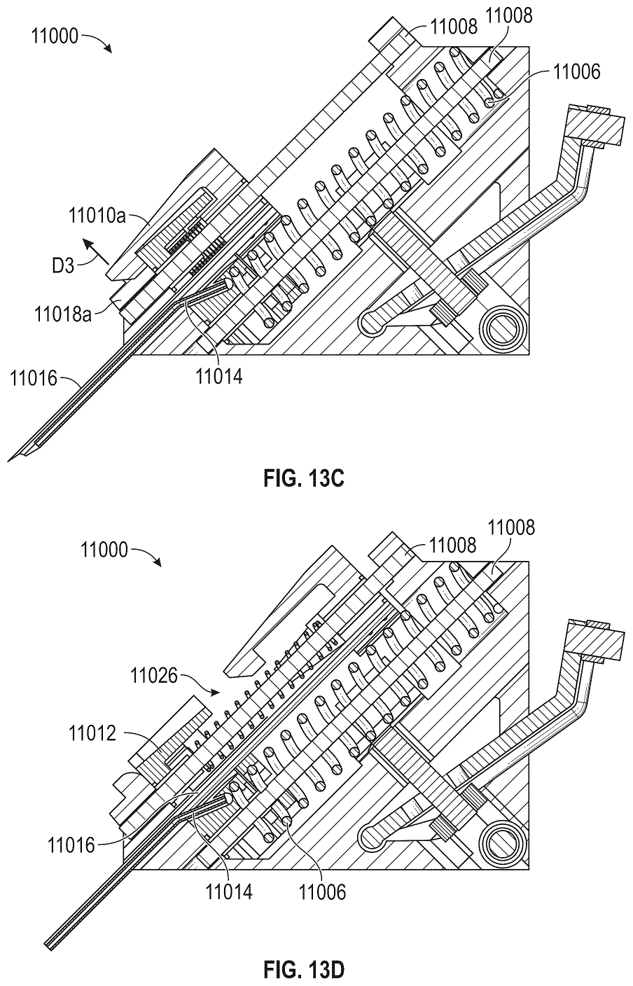

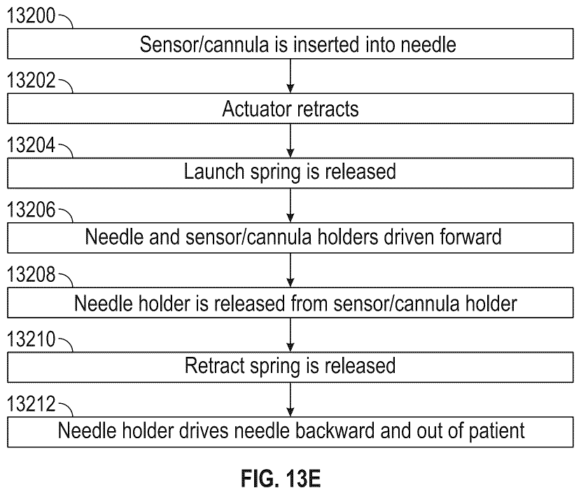

In some implementations, the roller may be coupled to one end of the trigger arm 10004. The other end of the trigger arm may be coupled to the body 10018 creating a hinge for the trigger arm to rotate about. The trigger arm 10004 may be further coupled to the trigger pin 10020, such that when the trigger arm 10004 rotates, the trigger pin 10020 is pulled outwards from the automated insertion device 10000, initiating a launch of the automated insertion device 10000. The launch of the automated insertion device 10000 will be described in more detail with respect to FIGS. 13A-13E.

The launch spring may engage one end with the body 10018 and another end with the sensor carrier 10012. When the automated insertion device 10000 is in a prelaunch configuration, the sensor carrier 10012 may be coupled to the needle carrier 10010. The retract spring 10026 may have one end engaged with the sensor carrier 10012 and another end engaged with the needle carrier 10010. The needle carrier 10010 may be coupled with the needle 10016 such that when the needle carrier 10010 moves, the needle 10016 moves as well. Similarly, the sensor carrier 10012 may be coupled with the an end of the sensor 10014 such that when the sensor carrier 10012 moves, the end of the sensor 10014 also moves and when the sensor carrier 10012 is stationary, the end of the sensor 10014 remains in place.

In some implementations, the needle 10016 is U-shaped (e.g., forms a U-shaped cavity), allowing the sensor 10014 to be fed into the a cavity in the needle 10016. For example, the needle 10016 may have a U-shaped cavity encompassing the sensor 10014. The body 10018 can include a sensor insertion opening 10015, as illustrated in FIG. 10C. As illustrated in FIG. 10D, the needle 10016 may have an open end facing the sensor 10014 and the sensor insertion opening 10015, allowing an end of the sensor 10014 to be inserted into the needle 10016 from the sensor insertion opening 10015.

In some implementations, the launch spring 10006 and the retract spring 10026 may comprise compression springs. When the automated insertion device 10000 is in a prelaunch configuration, the launch spring 10006 and the retract spring 10026 may be in a compressed position. The launch spring 10006 may be held in a compressed position by the sensor carrier 10012 and the trigger pin 10020 and the retract spring 10026 may be held in a compressed position by a locking mechanism on the needle carrier 10010. When the automated insertion device 10000 is in a postlaunch configuration, the launch spring 10006 may be extended along the guide rails 10008. However, in the extended state, the launch spring 10006 may maintain tension as to secure the sensor carrier 10012, thereby securing the sensor 10014 in an inserted position in the insertion site on the skin of the patient. Similarly, when the automated insertion device 10000 is in the postlaunch configuration, the retract spring 10026 may be extended along the guide rails 10008. The retract spring 10026 may maintain tension in the extended position as to secure the needle carrier 10010, thereby securing the needle 10016 in a retracted position out of the patient. The transition between the prelaunch and postlaunch position of the automated insertion device 10000 will be described in more detail with respect to FIGS. 13A-13E.

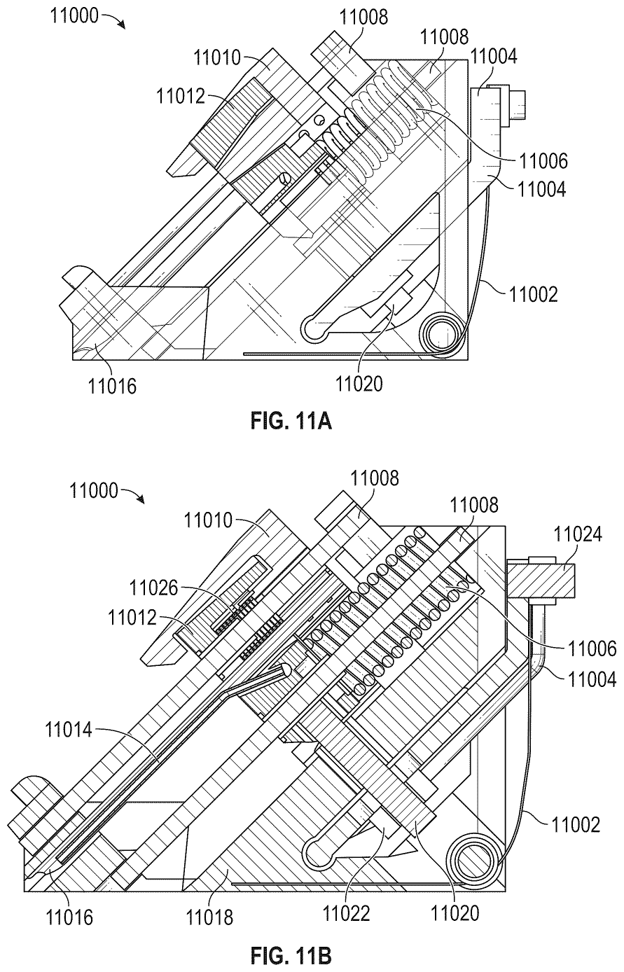

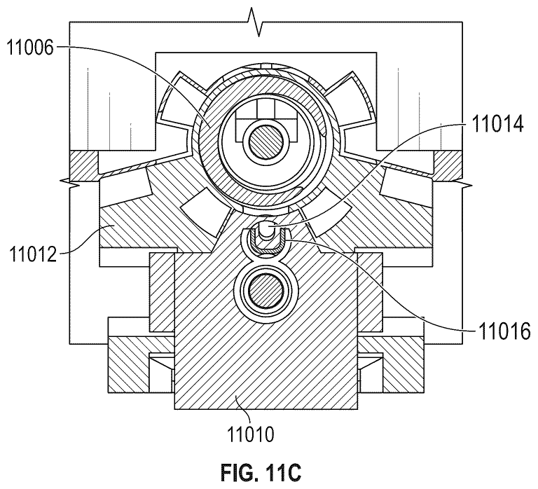

FIGS. 11A-11C illustrate an example automated insertion device 11000. In some implementations, the automated insertion device 11000 may be part of the modular disease management system as described above. For example, the automated insertion device 11000 may be used as the medication injector 2116 of FIGS. 2A-2F or the needle insertion device 4122 of FIGS. 4A-4P and/or in the combination sensor and medication injector module 3106 of FIG. 3. As described above, reducing the overall profile of the automated insertion device 11000 can improve patient comfort and maneuverability. As such, automated insertion device 11000 advantageously provides easy insertion of a cannula or other device into a patient, while maintaining a low profile. FIG. 11A illustrates a side view of the automated insertion device 11000. FIG. 11B illustrates a cross section side view the automated insertion device 11000.

The automated insertion device 11000 may be used to insert a cannula, such as the cannula 11014, or other device into an insertion site on the skin of a patient. In some implementations, the automated insertion device 11000 inserts the cannula at an angle at the insertion site on a patient. For example, the automated insertion device 11000 may insert the cannula into a patient at approximately a 45 degree angle, at an angle less than a 45 degrees, or at an angle above 45 degrees. The illustrated example contains a trigger arm 11004, a retracting wire 11002, a launch spring 11006, guide rails 11008, a needle carrier 11010, a cannula carrier 11012, a cannula 11014, a needle 11016, a body 11018, a trigger pin 11020, a stopper 11022, a roller 11024, and a retract spring 11026.

In some implementation, the cannula 11014 may be a cannula, catheter, or other device configured to deliver fluid, such as medication, at the insertion site on the skin of the patient. The automated insertion device 11000 may insert the cannula 11014 into the insertion site via the needle 11016. In various implementations, the needle 10016 can carry cannula 11014 into a patient. Then the automated insertion device 10000 may remove the needle 11016, leaving the cannula 11014 in the insertion site.

In various implementations, the automated insertion device 11000 may include many of the components of the automated insertion device 10000. For example, the retracting wire 11002 may be the same as the retracting wire 10002, the trigger arm 11004 may be the same as the trigger arm 10004, the launch spring 11006 may be the same as the launch spring 10006, the guide rails 11008 may be the same as the guide rails 10008, the needle carrier 11010 may be the same as the needle carrier 10010, the trigger pin 11020 may be the same as the trigger pin 10020, the stopper 11022 may the same as the stopper 10022, the roller 11024 may be the same as the roller 10024, and the retract spring 11026 may the same as the retract spring 10026.

In various implementations, the automated insertion device 11000 may have differing components that the automated insertion device 10000. For instance, like the sensor carrier 10012, the cannula carrier 11012 may be couple to the needle carrier 11010 in prelaunch configuration and be engaged to the retract spring 11026. However, the cannula carrier 11012 may be coupled with an end of the cannula 11014, rather than a sensor, such that when the cannula carrier 11012 moves, the end of the cannula 11014 also moves and when the cannula carrier 11012 is stationary, the end of the cannula 11014 remains in place. Further, like needle 10016, needle 11016 may be a U-shaped needle, allowing the cannula 11014 to be carried within the needle 11016. For example, the needle 11016 may have a U-shaped cavity encompassing the cannula 11014. However, in some implementations, the orientation of needle 11016 may differ from needle 10016. As illustrated in FIG. 11C, needle 11016 may be oriented to have an open end directed towards the launch spring 11006. Lastly, body 11018 may not have a sensor opening, such as sensor insertion opening 10015. Rather, automated insertion device 11000 can include a path for the cannula 11014 to be inserted into the needle 11016.

FIGS. 12A and 12B illustrate examples of configurations for retracting wires that can be used for an automated insertion device, such as an automated insertion device 10000 or an automated insertion device 11000. The configurations for the retracting wires may be utilized to engage the trigger arm of an automated insertion device to initialize the launch process.

FIG. 12A illustrates a side view and a back view of an automated insertion device 12000 with a retracting wire 12002 in a first configuration. Automated insertion device 12000 can correspond to automated insertion device 10000 or automated insertion device 11000. Automated insertion device 12000 can include a retracting wire 12002, a trigger arm 12004, a roller 12024, and anchor pin 12010. The retracting wire 12002 can correspond to the retracting wire 10002 or the retracting wire 11002, the trigger arm 12004 can correspond to the trigger arm 10004 or the trigger arm 11004, and the roller 12024 can correspond to the roller 10024 or the roller 11024.

In the illustrated example of FIG. 12A, the ends of the retracting wire 12002 are affixed to lower outer portions of the body of the automated insertion device 12000. The retracting wire 12002 loops around the roller 12024 and the anchor pin 12010. In some implementations, when the an electrical current is applied to the retracting wire 12002 the retracting wire 12002 contracts, decreasing in overall length. Because the retracting wire 12002 is affixed to the automated insertion device 12000, when the retracting wire 12002 contracts, a downward force is applied to the roller 12024, causing the trigger arm 12004 to rotate downward. The roller 12024 may be rounded and free to rotate to reduce friction and increase transfer force. The anchor pin 12010 may have an outer portion protruding from the body of the automated insertion device 12000 to hold the retracting wire 12002 in the desired configuration and may be rounded and/or rotate to further reduce friction.

FIG. 12B illustrates a side view and a bottom view of an automated insertion device 12050 with the retracting wire 12002 is a second configuration. The automated insertion device 12050 may be similar to the automated insertion device 12000 except the retracting wire 12002 may be affixed to an inner portion of the bottom body of the automated insertion device 12050 rather than the sides. As such, anchor pin 12052 may omit the outer portions of the anchor pin 12010.

While FIGS. 12A and 12B illustrate both ends of the retracting wire 12002 affixed to the body of an automated insertion device, other configurations are possible. For example, one end of the retracting wire 12002 may be affixed to another component, such as an external component, that applies a physical force to the retracting wire 12002 that is transferred to the trigger arm 12004 via the roller 12024.

FIG. 12C illustrates an example process for an automated needle insertion and needle removal device, such as automated insertion device 12000 or automated insertion device 12050, for initializing a launch of the automated needle insertion and needle removal device. At block 12200, a current is applied to a retracting wire. As described above, the retracting wire may comprise a material with shape memory, such as nitinol, that forms a set state when exposed to electrical current. As such, at block 12202, the retracting wire contracts in response to the electrical current.

At block 12204, the trigger arm rotates. As described above, when the retracting wire contracts, a force is applied on a roller and/or a trigger arm, pulling the arm downward. As will be described in more detail with respect to FIGS. 13A-13D, the trigger arm may be coupled to a trigger pin, such that when the trigger arm rotates, the force is applied to the trigger pin, pulling the trigger pin outwards. Further, at a first position, the trigger arm may be holding a needle carrier and a sensor or cannula carrier at a held position. As such, at block 12206 the trigger pin is moved from the first position and releases the needle carrier and sensor/cannula carrier from the held position. For example, at block 12206, the trigger pin may allow a launch spring, such as launch spring 10006, to push the needle carrier and sensor/cannula carrier forward, thereby initiating a launch of the automated needle insertion and needle removal device.

FIGS. 13A-13D illustrate an automated insertion and needle removal device at various positions while inserting a needle into and removing the needle from an insertion site of a patient to implant a cannula. While FIGS. 13A-13D illustrate automated insertion device 11000, other automated insertion devices, such as automated insertion device 10000 may operate in the same, or similar, manner. Further, all of the components described with respect to FIGS. 10A-10D, 11A-11C, and/or 12A and 12B may be present in FIGS. 13A-13D. FIG. 13A illustrates a cross section side view of an automated needle insertion and needle removal device prior to launch (also referred to as a prelaunch configuration). FIG. 13B illustrates a cross section side view of an automated needle insertion and needle removal device with a trigger arm rotated. FIG. 13C illustrates a cross section side view of an automated needle insertion and needle removal device with a needle in an inserted position. FIG. 13D illustrates a cross section side view of an automated needle insertion and needle removal device with a needle in a retracted position.

Referring to FIG. 13A, the launch spring 11006 and the retract spring 11026 are in a compressed state. The launch spring 11006 is held in place by a cannula carrier 11012 and the trigger pin 11020 at a first end 11020a. The retract spring 11026 is held in place by the needle carrier 11010. The needle carrier can include a latched portion 11010a that may release the 11010 from the cannula carrier, thereby releasing the retract spring 11026.

Referring to FIG. 13B, as a force is applied on the trigger arm 11004, such as by the process described above with respect to FIG. 12C, the trigger arm 11004 moves in direction D1. The trigger arm 11004 can be coupled to the trigger pin 11020 such that, as the trigger arm moves in direction D1, the trigger pin 11020 moves in direction D2. As the trigger pin 11020 moves in the direction D2, the first end 11020a may disengage the cannula carrier 11012, thereby releasing the launch spring 11006. The stopper 11022 may hold the trigger pin 11020 in a position with first end 11020a out of the travel path of the launch spring, the needle carrier 11010, and a cannula carrier 11012.

Referring to FIG. 13C, when the launch spring 11006 is released, the launch spring may push the cannula carrier 11012 and the needle carrier 11010 forward along the guide rails 11008, such that the needle 11016 and the cannula 11014 are inserted into an insertion site of a patient. As illustrated in FIG. 13C, the launch spring 11006 may maintain tension as to hold the cannula carrier 11012 in place. When the automated insertion device 11000 is in the fully inserted position illustrated in FIG. 13C, the latched portion 11010a of the needle carrier 11010 may interact with a protruded portion 11018a of the body 11018, forcing the latched portion 11010a in direction D3. As the latched portion 11010a moves in direction D3, the needle carrier 11010 is released from the cannula carrier 11012, thereby releasing the retract spring 11026. Referring to FIG. 13D, when the retract spring 11026 is released, the retract spring may push the needle carrier 11010 backward along the guide rails 11008, thereby removing the needle 11016 from the insertion site of the patient. The retract spring 11026 may also maintain tension such that the retract spring 11026 holds the needle carrier 11010 in a position and the needle 11016 remains fully retracted from the patient. As illustrated in FIG. 13D, the retract spring 11026 and the launch spring 11006 may secure the cannula carrier 11012 in place such that the cannula 11014 remains fixed in position in the insertion site of the patient.

FIG. 13E illustrates an example process for an automated needle insertion and needle removal device, such as automated insertion device 11000 or automated insertion device 10000, for inserting a cannula or a sensor into an insertion site of a patient. At block 13200 a sensor/cannula is inserted into a needle. In some implementations, a sensor, such as sensor 10014 is inserted into sensor insertion opening 10015 an into needle 10016. In some implementations, a cannula, such as needle 11016 is inserted into needle 11016.

At block 13202, an actuator retracts. In some implementations, the actuator may be a retracting wire and retract using the process described above with respect to FIG. 12C. In some implementation, the actuator may retract in response to a physical force applied to the actuator. In some implementations, the actuator can be connected to a trigger arm, such as trigger arm 11004, such that when the actuator retracts the trigger arm is moved in a downward direction (e.g., in direction D1).

At block 13204, a launch spring, such as launch spring 11006, is released. As described above, the actuator and/or trigger arm may be coupled to a release mechanism, such as trigger pin 11020. When the actuator retracts, the release mechanism may disengage a sensor holder, a cannula holder, or other component, such that the launch spring is released.

At block 13206, the needle and sensor/cannula holders are driven forward. When the launch spring is released, the launch spring can push the needle holder and the sensor/cannula holder forward along the guide rails 11008. The needle holder may be coupled to a needle, such as 11016, and the sensor/cannula holder may be coupled to a sensor or cannula (e.g., sensor 10014 or cannula 11014). As such, as the needle holder and the sensor/cannula holder are driven forward, a needle and a sensor/cannula are also driven forward and inserted into an insertion site of the patient.

At block 13208, the needle holder is released form the sensor or cannula holder. For example, as illustrated in FIG. 13C, a protruded portion 11018a of the body 11018 may engage the needle carrier 11010, releasing the needled carrier from the cannula carrier 11012. At block 13210, the retract spring is released. As described above, the retract spring may have one end engaged with the needle carrier and another end engaged with the cannula/sensor holder. As such, when the needler holder is released at block 13208, the retract spring is free to extend.

At block 13212, the needle holder drives needle backward out of the insertion site of the patient. As the retract spring decompresses and extends, the retract spring can force the needle holder backward along the guide rails. Since the needle holder is coupled to the needle, as the needle holder is forced backward, the needle is also forced backward, thereby removing the needle for the insertion site. As described above, after the needle holder drives the needle backward, the retract spring and the launch spring maintain tension, holding the sensor/cannula in place in the insertion site and the needle in place out of the insertion site.

### Terminology

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps, but may include additional steps. When used in the context of a device, the term “comprising” means that the device includes at least the recited features or components, but may also include additional features or components. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The term “and/or” as used herein has its broadest least limiting meaning which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Any methods disclosed herein need not be performed in the order recited. The methods disclosed herein include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within.

### EXAMPLES

Disclosed herein are additional examples of systems and methods described herein. Any of the examples in this disclosure may be combined in whole or in part. Any of the systems described in the examples may implement any of the methods, processes, and/or the like described herein and any of the methods described in the examples may be implemented by any of the systems described herein. Some aspects of the embodiments discussed above are disclosed in further detail in the additional examples, which are not in any way intended to limit the scope of the present disclosure. Those in the art will appreciate that many other embodiments also fall within the scope of the invention, as it is described herein above and in the claims. Any of the examples may include fewer or greater components or steps. Further, components and/or method steps described in the examples can be replaced with other components and/or method steps.

Example 1. A modular disease management system, comprising: a base configured to at least partially couple to skin of a patient; at least one module configured to removably couple to the base, the at least one module comprising: a first module, the first module comprising: a medication bladder; a medication pump configured to cause medication to flow from the medication bladder through a cannula configured to be inserted into a tissue site of the patient; and a cannula insertion device configured to insert the cannula into the tissue site of the patient by actuation of a spring released by a triggering component.

Example 2. The modular disease management system of Example 1, wherein the base comprises at least one physical interface configured to removably couple the at least one module.

Example 3. The modular disease management system of Example 2, wherein the at least one physical interface comprises a rigid portion of the base.

Example 4. The modular disease management system of Example 2, wherein the at least one physical interface forms a seal when the at least one module is couple to the base.

Example 5. The modular disease management system of Example 1, wherein the base comprises a flexible portion.

Example 6. The modular disease management system of Example 1, wherein the base comprises an electrical connector configured to electrically connect the at least one module to the base.

Example 7. The modular disease management system of Example 6, wherein the first module further comprises a printed circuit board (PCB), the PCB electrically coupled to at least the electrical connector and the medication pump.

Example 8. The modular disease management system of Example 7, wherein the PCB comprises a flex PCB.

Example 9. The modular disease management system of Example 1, wherein the at least one module further comprises a controller module, the controller module one or more computer processors.

Example 10. The modular disease management system of Example 9, wherein the controller module further comprises a battery.

Example 11. The modular disease management system of Example 10, wherein the battery is rechargeable.

Example 12. The modular disease management system of Example 9, wherein the controller module further comprises an antenna.

Example 13. The modular disease management system of Example 9, wherein the controller module is reusable and the base and the first module are disposable.

Example 14. The modular disease management system of Example 9, wherein the controller module is configured to be disconnected from the base and connected to a second base associated with a different disease management system.

Example 15. The modular disease management system of Example 1, wherein the at least one module further comprises a second module, the second module comprising: an analyte sensor configured to be inserted into a second tissue site of the patient; and a sensor insertion device configured to insert the analyte sensor into the second tissue site of the patient by actuation of a second spring released by a second triggering component.

Example 16. The modular disease management system of Example 15, wherein the analyte sensor is an electrochemical sensor.

Example 17. The modular disease management system of Example 15, wherein the analyte sensor is an optical sensor.

Example 18. The modular disease management system of Example 15, wherein the at least one module further comprises a combined module configured to house the first module and second module.

Example 19. The modular disease management system of Example 15, wherein information from the analyte sensor is communicated to one or more electronic components.

Example 20. The modular disease management system of Example 19, wherein based on the information from the analyte sensor, the one or more electronic components are configured to cause the medication pump to: increase a medication dose flowing in the cannula, decrease the medication dose flowing in the cannula, or maintain the medication dose flowing in the cannula.

Example 21. The modular disease management system of Example 1, wherein the cannula insertion device comprises a needle associated with the cannula.

Example 22. The modular disease management system of Example 21, wherein the needle comprises a U-shaped cavity encompassing the cannula.

Example 23. The modular disease management system of Example 21, wherein to insert the cannula into the tissue site of the patient, the needle is inserted along with the cannula into tissue site by the actuation of the spring.

Example 24. The modular disease management system of Example 23, wherein the cannula insertion device is further configured to remove the needle from the tissue site of the patient by actional of a second spring.

Example 25. The modular disease management system of Example 1, wherein the cannula is inserted into the tissue site of the patient at approximately a 45 degree angle.

Example 26. The modular disease management system of Example 1, wherein the triggering component comprises: a trigger pin configured to release the spring when actuated by a trigger arm; and a retracting wire configured to contract, causing the trigger arm to actuate the trigger pin.

Example 27. The modular disease management system of Example 26, wherein the retracting wire comprises a nitinol wire.

Example 28. The modular disease management system of Example 27, wherein the nitinol wire is configured to contract in response to an electrical current received from one or more electronic components.

Example 29. The modular disease management system of Example 28, wherein the base comprises the one or more electronic components.

Example 30. The modular disease management system of Example 1, wherein the medication pump comprises an insulin pump and the medication bladder comprises insulin.

Example 31. A disease management system, comprising: a medication bladder; a medication pump configured to cause medication to flow from the medication bladder through a cannula configured to be inserted into a tissue site of a patient; a cannula insertion device configured to insert the cannula into the tissue site of the patient, the cannula insertion device comprising: a needle; at least one spring; and a triggering component comprising: a trigger pin configured to release a first spring when actuated by a trigger arm; and a nitinol wire configured to contract in response to an electrical signal, causing the trigger arm to actuate the trigger pin; wherein the first spring, when released, is configured to insert the cannula into the tissue site of the patient; and a controller comprising one or more processors electrically coupled to the cannula insertion device and configured to transmit the electrical signal to the nitinol wire.

Example 32. The disease management system of Example 31, wherein the controller is configured to receive a user input and transmit the electrical signal to the nitinol wire in response to the user input.

Example 33. The disease management system of Example 31, further comprising: a sensor insertion device configured to insert an analyte sensor a second tissue site of the patient, the sensor insertion device comprising: a second needle; at least a second spring; and a second triggering component comprising: a second trigger pin configured to release a first spring when actuated by a second trigger arm; and a second nitinol wire configured to contract in response to a second electrical signal, causing the second trigger arm to actuate the second trigger pin; wherein the second spring, when released, is configured to insert the analyte sensor into the tissue site of the patient.

Example 34. The disease management system of Example 33, wherein the analyte sensor is an electrochemical sensor.

Example 35. The disease management system of Example 33, wherein the analyte sensor is an optical sensor.

Example 36. The disease management system of Example 33, wherein information from the analyte sensor is communicated to the controller.

Example 37. The disease management system of Example 36, wherein based on the information from the analyte sensor, the controller is further configured to cause the medication pump to: increase a medication dose flowing in the cannula, decrease the medication dose flowing in the cannula, or maintain the medication dose flowing in the cannula.

Example 38. The disease management system of Example 33, wherein the needle comprises a U-shaped cavity encompassing the cannula.

Example 39. The disease management system of Example 33, wherein the cannula is inserted into the tissue site of the patient at approximately a 45 degree angle.

Example 40. An automatic insertion device, comprising: a needle configured to insert and/or emplace a device at a tissue site of a patient when actuated; one or more springs configured to actuate the needle when released; and a triggering component configured to release the one or more springs, the triggering component comprising: a trigger pin configured to release at least a first spring of the one or more springs when actuated by a trigger arm; and a nitinol wire configured to contract in response to an electrical signal, causing the trigger arm to actuate the trigger pin; wherein upon the nitinol wire a contracting, the triggering component releases at least the first spring, the first spring actuates the needle, and the device is emplaced at the tissue site of the patient.

Example 41. The automatic insertion device of Example 40, wherein the device is a continuous glucose monitor.

Example 42. The automatic insertion device of Example 40, wherein the device is a cannula connected to an insulin pump.

Example 43. The automatic insertion device of Example 40, wherein the needle is inserted into the patient at approximately a 45 degree angle.

Example 44. A method for automatically inserting a device into a patient, the method comprising: inserting a cannula into a needle configured to emplace the cannula when the needle is inserted at a tissue site of a patient; activating, using an electrical signal, a triggering component configured to release one or more springs, at least a first spring of the one or more springs configured to insert the needle into the tissue site, the triggering component comprising: a trigger pin configured to release at least the first spring when actuated by a trigger arm; and a nitinol wire configured to contract in response to the electrical signal, causing the trigger arm to actuate the trigger pin; and administering, using a medication pump coupled to the cannula, a medication to the patient via the cannula.

Example 45. The method of Example 44, wherein the medication comprises insulin.

Example 46. The method of Example 44, wherein the needle is inserted into the patient at approximately a 45 degree angle.

Example 47. A modular disease management system, comprising: a base configured to at least partially couple to skin of a patient; at least one module configured to removably couple to the base, the at least one module comprising: a first module, the first module comprising: an analyte sensor configured to be inserted into a tissue site of the patient; and a sensor insertion device configured to insert the analyte sensor into the tissue site of the patient by actuation of a spring released by a triggering component.

Example 48. A disease management system, comprising: a controller comprising one or more processors; a needle insertion device comprising a needle and one or more springs; and a base configured to house the controller and the needle insertion device; wherein the needle insertion device is configured to initiate a launch of the one or more springs to drive a needle into an insertion site of a patient based on a control signal received from the controller.

Example 49. The disease management system of Example 48, further comprising a continuous glucose monitor (CGM), wherein needle insertion device is further configured to insert an end of the CGM into the insertion site.

Example 50. The disease management system of Example 49, wherein the needle insertion device is further configured to remove the needle from the insertion site while the CGM remains.

Example 51. The disease management system of Example 48, further comprising an insulin pump and a cannula coupled to the insulin pump, wherein needle insertion device is further configured to insert an end of the cannula into the insertion site.

Example 52. A modular disease management system, comprising: a base configured to receive a plurality of modules, the base comprising: an electrical connector; an adhesive layer; and at least one physical interface, wherein each physical interface is configured to couple the plurality of modules to a surface of the base and to provide an electrical signal to the plurality of modules via the electrical connector; wherein, the plurality of modules comprise: a controller module; a pump module, wherein the pump module comprises a pump and a pouch assembly containing a substance, wherein the pouch assembly is configured to connect with the pump; at least one sensor module; and at least one medication module; and wherein, the controller module is configured to activate the pump such that the pump draws the substance from the pouch assembly into the medication module.

Example 53. The modular disease management system of Example 52, wherein the base, the at least one sensor module, the at least on medication module, and the pump module are disposable.

Example 54. The modular disease management system of Example 52, wherein the at least one sensor module comprises a continuous glucose monitor injector and the at least one medication module comprises an insulin injector.

Example 55. The modular disease management system of Example 54, wherein the continuous glucose monitor injector is configured to send a glucose reading to the controller module.

Example 56. The modular disease management system of Example 54, wherein the continuous glucose monitor injector and the insulin injector are housed in a single automatic insertion module.

Example 57. The modular disease management system of Example 52, wherein the controller module comprises a battery and a control unit.

Example 58. The modular disease management system of Example 57, wherein the battery is configured to allow for volume expansion.

Example 59. The modular disease management system of Example 57, wherein the controller module comprises at least one cutout for a high profile component attached to the control unit.

Example 60. The modular disease management system of Example 52, wherein the controller module is configured to communicate with a user terminal.

Example 61. The modular disease management system of Example 52, wherein the electrical connector is a flexible cable.

Example 62. The modular disease management system of Example 61, wherein the flexible cable is 0.31 millimeters thick.

Example 63. The modular disease management system of Example 52, wherein the controller module comprises a compartment seal configured to interface with the physical interface to create an electrical contact with the electrical connector and prevent water ingress into the controller module.

Example 64. An automatic insertion device for inserting a cannula into a patient, comprising: a housing; a needle associated with a cannula positioned in the housing; a launch spring holder configured to house a launch spring, the launch spring configured to push the needle and cannula forward; a first trigger release arm configured to apply an opposing force on the launch spring holder such that the launch spring is in a hold position; and a second trigger release arm coupled to the first trigger release arm, the second trigger release arm further coupled to an actuator configured to contract in response to an electrical signal such that the first trigger release arm and the second trigger release arm withdraw when the actuator contracts; and wherein, upon the withdrawal of the first trigger release arm and the second trigger release, the opposing force on the launch spring holder is disengaged such that the launch spring pushes the needle and cannula forward to allow for the needle and the cannula to insert into a patient.

Example 65. The automatic insertion device of Example 64, wherein the actuator is a nitinol wire.

Example 66. The automatic insertion device of Example 64, wherein the device further comprises at least one guiding rail located at a top of the housing.

Example 67. The automatic insertion device of Example 64, wherein the first trigger release arm comprises a rectangular carve out configured to allow the second trigger release arm to connect to the first trigger release arm.

Example 68. The automatic insertion device of Example 67, wherein the second trigger release arm comprises an end that is circular in shape, such that the second trigger release arm is held under tension by the rectangular carve out prior to the actuator contracting and the second trigger release is able to freely rotate with the rectangular carve out during the actuator contracting.

Example 69. The automatic insertion device of Example 64, wherein the second trigger release arm is rectangular in shape.

Example 70. The automatic insertion device of Example 64, wherein the needle is U-shaped such that the cannula is located inside the U-shape of the needle.

Example 71. The automatic insertion device of Example 64, further comprising: a retract spring holder configured to house a retract spring in a hold position; and a release switch configured to release the retract spring holder; wherein, upon the insertion of the needle and cannula into the patient, the release switch releases the retract spring holder such that the retract spring removes the needle from the patient while the cannula remains inserted into the patient.

Example 72. An automatic insertion device for inserting an analyte sensor into a patient, comprising: a housing; a needle associated with an analyte sensor positioned in the housing; a launch spring holder configured to house a launch spring, the launch spring configured to push the needle and the analyte sensor forward; a first trigger release arm configured to apply an opposing force on the launch spring holder such that the launch spring is in a hold position; and a second trigger release arm coupled to the first trigger release arm, the second trigger release arm further coupled to an actuator configured to contract in response to an electrical signal such that the first trigger release arm and the second trigger release arm withdraw when the actuator contracts; and wherein, upon the withdrawal of the first trigger release arm and the second trigger release, the opposing force on the launch spring holder is disengaged such that the launch spring pushes the needle and cannula forward to allow for the needle and the cannula to insert into a patient.

Example 73. A method for automatically inserting a cannula into a patient, the method comprising: preloading a launch spring configured to push a needle and a cannula forward along a fixed axis, wherein the launch spring is housed in a launch spring holder and wherein a first trigger release arm is configured to apply an opposing force on the launch spring holder such that the launch spring is in a hold position, wherein a second trigger release arm is coupled to the first trigger release arm, and the second trigger release arm is further coupled to an actuator configured to contract in response to an electrical signal such that the first trigger release arm and the second trigger release arm withdraw when the actuator contracts, and wherein, upon the withdrawal of the first trigger release arm and the second trigger release, the opposing force on the launch spring holder is disengaged such that the launch spring pushes the needle and cannula forward to allow for the needle and the cannula to insert into the patient; and causing the actuator to retract.

Example 74. The method of Example 73 further comprising: preloading a retract spring configured to push the needle backward along the fixed axis, wherein the retract spring is housed in a retract spring holder and wherein a retract release trigger is configured to apply an opposing force on the retract spring holder such that the retract spring is in a hold position; wherein, upon the insertion of the needle and cannula into the patient, the retract release trigger releases the retract spring holder such that the retract spring removes the needle from the patient while the cannula remains inserted into the patient.

Example 75. A method for automatically inserting an analyte sensor into a patient, the method comprising: preloading a launch spring configured to push a needle and an analyte sensor forward along a fixed axis, wherein the launch spring is housed in a launch spring holder and wherein a first trigger release arm is configured to apply an opposing force on the launch spring holder such that the launch spring is in a hold position, wherein a second trigger release arm is coupled to the first trigger release arm, and the second trigger release arm is further coupled to an actuator configured to contract in response to an electrical signal such that the first trigger release arm and the second trigger release arm withdraw when the actuator contracts, and wherein, upon the withdrawal of the first trigger release arm and the second trigger release, the opposing force on the launch spring holder is disengaged such that the launch spring pushes the needle and the analyte sensor forward to allow for the needle and the analyte sensor to insert into the patient; and causing the actuator to retract.

Example 76. The method of Example 75 further comprising: preloading a retract spring configured to push the needle backward along the fixed axis, wherein the retract spring is housed in a retract spring holder and wherein a retract release trigger is configured to apply an opposing force on the retract spring holder such that the retract spring is in a hold position; wherein, upon the insertion of the needle and cannula into the patient, the retract release trigger releases the retract spring holder such that the retract spring removes the needle from the patient while the analyte sensor remains inserted into the patient.

Example 77. An automatic insertion device, comprising: a housing; a needle associated with a cannula located within the housing; at least three torsion springs configured to connect to an actuator located within the housing; at least three spring holders configured to preload each torsion spring; and the actuator configured to release the at least three torsion springs upon a trigger.

Example 78. The automatic insertion device of Example 77, wherein the at least three torsion springs comprises: a first spring; a second spring; and a third spring, wherein the actuator is configured to release the first spring which triggers the release of the second spring and then releases the needle and cannula forward to insert into a patient, wherein the third spring is configured to retrieve the needle from insertion, and wherein the third spring is further configured to hold the needle at a high position.

Example 79. The automatic insertion device of Example 77, wherein a guide rail holds the needle and the cannula at an angle.

Example 80. The automatic insertion device of Example 77, wherein the actuator is a nitinol wire.

Example 81. The automatic insertion device of Example 77, wherein the actuator is an electrical actuator.

Example 82. The automatic insertion device of Example 77, wherein the at least torsion springs remain in a locked position until the trigger.

Example 83. A method for automatically inserting a cannula into a patient, the method comprising: preloading a first torsion spring, wherein the first torsion spring is held at tension by a first spring holder and a trigger release holder; preloading a second torsion spring, wherein the second torsion spring is held at tension by a second spring holder and a needle holder, wherein the needle holder is held in place by a trigger; preloading a third torsion spring, wherein the third torsion spring is held at tension by a third spring holder and a guiderail; and causing the first torsion spring to disengage from the trigger release holder, wherein, upon the first torsion spring disengaging from the trigger release holder, the first torsion spring causes the trigger to rotate and disengage from the second torsion spring, wherein, upon the trigger disengaging from the second torsion spring, the second torsion spring drives the needle holder forward along the guiderail and a needle into the patient, wherein, upon the needle being inserted into the patient, the third torsion spring is disengaged from the guiderail, wherein, upon the third torsion spring disengaging from the guiderail, the third torsion spring drives the needle holder backward along the guiderail and the needle out of the patient.

Example 84. A method for automatically inserting a cannula into a patient, the method comprising: preloading a launch spring configured to push needle carrier, coupled to a needle, and a cannula carrier, coupled to a cannula, forward along at least one guiderail, wherein the launch spring is coupled to a launch spring holder configured to rotate in response to a downward force and wherein a trigger release is configured to apply an opposing force on the launch spring holder such that the launch spring is in a hold position and the launch spring holder is in a hold position, wherein the trigger release is coupled to an actuator configured to contract in response to an electrical signal such that the trigger release withdraws when the actuator contracts, wherein, upon the withdrawal of the trigger release, the opposing force on the launch spring holder is disengaged such that the launch spring pushes the needle carrier and cannula carrier forward along the at least one guiderail to allow for the needle and the cannula to insert into the patient; and causing the actuator to retract.

Example 85. The method of Example 84 further comprising: preloading a retract spring configured to push the needle carrier backward along the at least one guiderail, wherein the retract spring is housed in a retract spring holder and wherein a retract release trigger is configured to apply an opposing force on the retract spring holder such that the retract spring is in a hold position; wherein, upon the insertion of the needle and cannula into the patient, a rotator spring applies a force on the launch spring holder such that the launch spring holder rotates, wherein, upon the launch spring holder rotating, the launch spring pushes the needle carrier such that the needle carrier disengages with the at least one guiderail, and wherein, upon the needle carrier disengaging from the at least one guiderail, the retract release trigger releases the retract spring holder such that the retract spring pushes the needle carrier backward and the needle out of the patient.

Example 86. An automatic insertion device for inserting a cannula into a patient, comprising: a housing; a needle associated with a cannula positioned in the housing; at least one guide rail; a launch spring holder configured to house a launch spring, the launch spring configured to push the needle and cannula forward along the guide rail; a trigger release configured to hold the launch spring in a tension state; an actuator coupled to the trigger release, the actuator configured to retract the trigger release; and where, upon the actuator retracting the trigger release, the trigger release disengages from the launch spring such that the launch spring pushes the needle and cannula forward to allow for the needle and the cannula to insert into a patient.

Example 87. The automatic insertion device of Example 86, wherein the launch spring holder is further configured to rotate within the housing.

Example 88. The automatic insertion device of Example 87, further comprising a stamp sheet metal spring configured to cause the launch spring holder to rotate into alignment with the guide rail.

Example 89. The automatic insertion device of Example 86, further comprising: a retraction spring configured to engage with the needle; wherein, upon the insertions of the needle and the cannula into a patient, the retraction spring is released such that the needle is pushed backward along the guide rail and retracted from the patient.

Example 90. The automatic insertion device of Example 86, wherein the at least one guide rail comprises a guide wire.

Example 91. The automatic insertion device of Example 89, wherein the retraction spring is an elastic band.

1. A modular disease management system, comprising:
a base configured to at least partially couple to skin of a patient; and
at least one module configured to removably couple to the base, the at least one module comprising:
a first module, the first module comprising:
a medication bladder;
a medication pump configured to cause medication to flow from the medication bladder through a cannula configured to be inserted into a tissue site of the patient; and
a cannula insertion device configured to insert the cannula into the tissue site of the patient by actuation of a spring released by a triggering component.

2. The modular disease management system of claim 1, wherein the base comprises at least one physical interface configured to removably couple the at least one module.

3. The modular disease management system of claim 2, wherein the at least one physical interface comprises a rigid portion of the base.

4. The modular disease management system of claim 2, wherein the at least one physical interface forms a seal when the at least one module is couple to the base.

5. The modular disease management system of claim 1, wherein the base comprises a flexible portion.

6. The modular disease management system of claim 1, wherein the base comprises an electrical connector configured to electrically connect the at least one module to the base.

7. The modular disease management system of claim 6, wherein the first module further comprises a printed circuit board (PCB), the PCB electrically coupled to at least the electrical connector and the medication pump.

8. The modular disease management system of claim 7, wherein the PCB comprises a flex PCB.

9. The modular disease management system of claim 1, wherein the at least one module further comprises a controller module, the controller module one or more computer processors.

10. (canceled)

11. (canceled)

12. (canceled)

13. The modular disease management system of claim 9, wherein the controller module is reusable and the base and the first module are disposable.

14. The modular disease management system of claim 9, wherein the controller module is configured to be disconnected from the base and connected to a second base associated with a different disease management system.

15. The modular disease management system of claim 1, wherein the at least one module further comprises a second module, the second module comprising:
an analyte sensor configured to be inserted into a second tissue site of the patient; and
a sensor insertion device configured to insert the analyte sensor into the second tissue site of the patient by actuation of a second spring released by a second triggering component.

16. The modular disease management system of claim 15, wherein the analyte sensor is an electrochemical sensor.

17. The modular disease management system of claim 15, wherein the analyte sensor is an optical sensor.

18. (canceled)

19. (canceled)

20. (canceled)

21. The modular disease management system of claim 1, wherein the cannula insertion device comprises a needle associated with the cannula.

22. The modular disease management system of claim 21, wherein the needle comprises a U-shaped cavity encompassing the cannula.

23. The modular disease management system of claim 21, wherein to insert the cannula into the tissue site of the patient, the needle is inserted along with the cannula into tissue site by the actuation of the spring.

24. The modular disease management system of claim 23, wherein the cannula insertion device is further configured to remove the needle from the tissue site of the patient by actional of a second spring.

25. The modular disease management system of claim 1, wherein the cannula is inserted into the tissue site of the patient at approximately a 45 degree angle.

26. The modular disease management system of claim 1, wherein the triggering component comprises:
a trigger pin configured to release the spring when actuated by a trigger arm; and
a retracting wire configured to contract, causing the trigger arm to actuate the trigger pin.

27. The modular disease management system of claim 26, wherein the retracting wire comprises a nitinol wire.

28-46. (canceled)
